(19) United States
(12) Patent Application Publication
(10) Pub. No.: US 2007/0212675 A1
(43) Pub. Date: Sep. 13, 2007

(54) Flap Endonuclease (Fen1) Regulatory Sequences And Uses Thereof

(75) Inventors: Paul Hallenbeck, Chester Springs, PA (US); Garret Hampton, San Diego, CA (US); Carl Hay, Damascus, MD (US); Ying Huang, Oleny, MD (US); John Jakubczak, East Lyme, CT (US); Sandrina Phipps, Reston, VA (US)

Correspondence Address: DLA PIPER US LLP, 153 TOWNSEND STREET, SUITE 800, SAN FRANCISCO, CA, 94107-1957, US

(73) Assignees: NOVARTIS AG, Basel (CH); IRM LLC, Hamilton (BM)

(21) Appl. No.: 10/553,459

(22) PCT Filed: Apr. 15, 2004
(86) PCT No.: PCT/US04/11622

§ 371 Date: Mar. 9, 2007

Related U.S. Application Data
(60) Provisional application No. 60/463,148, filed on Apr. 15, 2003.

(51) Int. Cl. C12Q 1/00

(52) U.S. Cl. 435/4

(57) ABSTRACT

FEN1 transcriptional regulatory sequences (TREs) are provided. FEN1 TREs provide for transcriptional regulation dependent upon transcription factors that are only active in cancer cells. The FEN1 TREs may be used as a vehicle for introducing new genetic capability, particularly associated with cytotoxicity, for selective expression in cancer cells.

This application is related to Provisional U.S. Patent Application Ser. No. 60/463148, filed Apr. 15, 2003, which is incorporated herein by reference in its entirety.

### FIELD OF THE INVENTION

The invention relates to novel flap endonuclease 1 (FEN1) regulatory sequences useful for cancer cell specific gene expression. The invention further relates to vector compositions comprising FEN1 regulatory sequences and methods for their use in therapy of cancer.

### BACKGROUND OF THE INVENTION

Currently, standard medical treatments for treatment of cancer including chemotherapy, surgery, radiation therapy and cellular therapy, have clear limitations with regard to both efficacy and toxicity. To date, these approaches have met with varying degrees of success dependent upon the type of cancer, general health of the patient and stage of disease at the time of diagnosis. Improved strategies that combine these standard medical treatments with novel approaches may provide a means for enhanced efficacy and decreased toxicity. A major, indeed the overwhelming, obstacle to cancer therapy is the problem of selectivity, that is, the ability to inhibit the multiplication of tumor cells, while leaving unaffected the function of normal cells. The therapeutic ratio, or ratio of tumor cell killing to normal cell killing of traditional tumor chemotherapy, is only about 1.5:1. Thus, more effective treatment methods and pharmaceutical compositions for therapy and prophylaxis of cancer are needed.

Vector-mediated gene delivery forms the basis of an innovative and potentially powerful disease-fighting tool in which an exogenous nucleotide is provided to a cell by way of a delivery vehicle such as a viral or non-viral vector. This approach holds great potential in treating not only many forms of cancer, but other diseases as well.

A number of vectors have been described as both vehicles for gene therapy and as candidate anticancer agents. An adenoviral vector containing the gene for p53 (which is mutated or inactivated in many cancers such as head and neck squamous cell carcinoma) has recently been approved for gene therapy of cancer in China. (New Scientist, 2003). Adenovirus has emerged as a virus that can be engineered with oncotropic properties. See, for example, U.S. Pat. No. 5,747,469; U.S. Pat. No. 5,801,029; U.S. Pat. No. 5,846,945; U.S. Pat. No. 5,747,469; WO 99/59604; WO 98/35554; WO 98/29555; U.S. Pat. Nos. 6,638,762; and 6,676,935. Specific attenuated replication-competent viral vectors have been developed for which selective replication in cancer cells destroys those cells. For example, various cell-specific replication-competent adenovirus vectors, which preferentially replicate (and thus destroy) certain cell types, are described, for example, in WO 95/19434, WO 98/39465, WO 98/39467, WO 98/39466, WO 99/06576, WO 98/39464, WO 00/15820. Improving the delivery of these vectors, both to local-regional and disseminated disease, as well as improving the vectors to promote intratumoral spread is of particular interest.

Flap endonuclease 1 (FEN1) was originally isolated as a DNA structure-specific endonuclease that cleaves a flap strand of branched DNA with a 5′ single-stranded terminus at the position near its junction to the double-stranded structure (Harrington and Lieber, EMBO J, 1994, 13:1235-1246). Subsequently, it was found to be identical or homologous to previously isolated proteins DnaseIV (Lindahl, Eur J Biochem, 1991, 18:407-414), pL (Guggenheimer et al., J Biol Chem, 1984, 259:7815-7825), 5′→3′ exonuclease (Siegal et al., Proc Natl Acad Sci USA, 1992, 89:9377-9381), and MF1 (Waga et al., J Biol Chem, 1994, 269:10923-10934). Thus FEN1 carries several distinct nuclease activities on specific structured DNA substrates. It works as an endonuclease on 5′-flap structured DNA, as a 5′→3′ exonuclease on nicked or gapped dsDNA (Siegal et al., Proc Natl Acad Sci USA, 1992, 89:9377-9381; Harrington and Lieber, J Biol Chem, 1995, 270:45034508), and as a ribonuclease on RNA-primed Okazaki fragments generated during discontinuous DNA replication (Waga et al., J Biol Chem, 1994, 269:10923-10934; (Turchi et al., Proc Natl Acad Sci USA, 1994, 91:9803-9807). In addition, FEN1 has an activity for removing 5′-incised AP sites (Price and Lindahl, Biochemistry, 1991, 30:8631-8637; DeMott et al., J Biol Chem, 1996, 271:30068-30076). Furthermore, recent studies demonstrate that PCNA directly binds to FEN1 and stimulates its activity (Li et al., J Biol Chem, 1995, 270:22109-22112; Wu et al., Nucleic Acids Res, 1996, 24:2036-2043).

Colon cancer is malignant tissue that grows in the wall of the colon. The majority of tumors begin when normal tissue in the colon wall forms an adenomatous polyp, or pre-cancerous growth projecting from the colon wall. As this polyp grows larger, a tumor is formed. The process can take many years, with the risk of colon cancer rising substantially after age 50, but every year there are numerous cases in younger people. The stage of cancer tells how far the tumor has invaded the colon wall, and if it has spread to other parts of the body. At stage 0 (also called carcinoma in situ), the cancer is confined to the outermost portion of the colon wall. At stage I, the cancer has spread to the second and third layer of the colon wall, but not to the outer colon wall or beyond. This is also called Dukes' A colon cancer. At stage II, the cancer has spread through the colon wall, but has not invaded any lymph nodes. This is also called Dukes' B colon cancer. At stage II the cancer is metastatic, and has spread through the colon wall and into lymph nodes, but has not spread to other areas of the body. This is also called Dukes' C colon cancer. At stage IV, the cancer has spread to other areas of the body, e.g. liver, lungs, etc. This is also called Dukes' D colon cancer.

Forty to fifty percent of patients have metastatic disease at the time of diagnosis, or have a recurrence of the disease after therapy. The prognosis for these patients is poor with conventional therapy, which is fluorouracil, Leucovorin, and irinotecan. With this therapy, an average of 39% of patients have a response, but the average survival time is only 15 months.

Although current therapies have met with some success in the treatment of local and disseminated cancer, there remains a need for improved therapeutic regimens that specifically target cancer, such as colon cancer. There is therefore, substantial interest in the development of improved vectors, which target cancer in vivo.

### SUMMARY OF THE INVENTION

The present invention provides an isolated nucleic acid sequence comprising a cancer specific transcriptional regulatory element (TRE) derived from the sequence upstream of the translational start codon of a Flap endonuclease 1 (FEN1) gene, wherein the TRE is specific for cancer cells.

In one aspect, the FEN1TRE comprises a nucleotide sequence selected from the group consisting of: (a) the 2259 bp sequence shown in SEQ ID NO:1; (b) a fragment of the 2259 bp sequence shown in SEQ ID NO: 1, wherein the fragment has tumor selective transcriptional regulatory activity; (c) a nucleotide sequence having at least 80, 85, 90, 91, 92, 93, 94, 95, 96, 97, 98, 99% or more % identity over its entire length to the 2259 bp sequence shown in SEQ ID NO: 1 when compared and aligned for maximum correspondence, as measured using a standard sequence comparison algorithm or by visual inspection, wherein the nucleotide sequence has tumor selective transcriptional regulatory activity; and (d) a nucleotide sequence having a full-length complement that hybridizes under stringent conditions to the 2259 bp sequence shown in SEQ ID NO:1, wherein the nucleotide sequence has tumor selective transcriptional regulatory activity.

In a related aspect, the invention provides a gene delivery vector comprising a FEN1 TRE. The gene delivery vector may be a replication competent adenovirus vector which selectively replicates in cancer cells.

In one embodiment, a replication competent adenovirus vector of the invention has a first and optionally a second adenovirus gene essential for replication under transcriptional control of a FEN1 TRE, wherein the first and second adenoviral genes may co-transcribed by way of an IRES or a self-processing cleavage sequence, such as a 2A sequence.

In another embodiment, a replication competent adenovirus vector of the invention has a second adenovirus gene essential for replication under transcriptional control of a plasminogen activator urokinase (uPA) TRE, a PRL-3-TRE, a TERT-TRE or an E2F-TRE.

In a further embodiment, a replication competent adenovirus vector of the invention comprises a transgene.

The invention also provides a method for selective cytolysis of cancer cells by administering a vector comprising a FEN1 TRE having a nucleotide sequence presented as SEQ ID NO:1 or a fragment of the sequence shown in SEQ ID NO: 1, wherein upon introduction into the cell, the vector effects selective cytolysis of tumor cells.

### DESCRIPTION OF THE DRAWINGS

FIG. 1 shows the results of a vector production assay for Ar13pAFenF. Vector production was measured from infected cells at 10 MOI and harvested three days post-infection. Biological titer was determined by limiting dilution and presented as mean +sd of triplicate CVLs.

### DETAILED DESCRIPTION OF THE INVENTION

The invention provides novel FEN1 transcriptional regulatory elements (TREs) which preferentially enhance the net transcription of operably-linked cis transcription units in cancer cells. The TREs of the present invention are preferentially active in cancer cells as compared with other tissues. The invention also provides compositions comprising a FEN1 TRE of the invention for therapy of hyperplasia and neoplasia, and methods for selective cytolysis of cancer cells using the same. The compositions and methods of the invention rely on the use of polynucleotides comprising a FEN1 TRE, suitable for use as gene-targeting constructs and/or for the expression of transgenes. In one aspect the invention provides a vector, e.g. a viral vector, comprising a FEN1 TRE of the invention.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991).

### DEFINITIONS

Unless otherwise indicated, all terms used herein have the same meaning as they would to one skilled in the art and the practice of the present invention will employ, conventional techniques of microbiology and recombinant DNA technology, which are within the knowledge of those of skill of the art.

As used herein, the terms “neoplastic cells”, “neoplasia”, “tumor”, “tumor cells”, “carcinoma”, “carcinoma cells”, “cancer” and “cancer cells”, (used interchangeably) refer to cells which exhibit relatively autonomous growth, so that they exhibit an aberrant growth phenotype characterized by a significant loss of control of cell proliferation. Neoplastic cells can be malignant or benign.

As used herein, “suppressing tumor growth” refers to reducing the rate of growth of a tumor, halting tumor growth completely, causing a regression in the size of an existing tumor, eradicating an existing tumor and/or preventing the occurrence of additional tumors upon treatment with the compositions, kits or methods of the present invention. “Suppressing” tumor growth indicates a growth state that is curtailed when compared to growth without intervention using the cancer-specific vectors of the invention. Tumor cell growth can be assessed by any means known in the art, including, but not limited to, measuring tumor size, determining whether tumor cells are proliferating using a ³H-thymidine incorporation assay, or counting tumor cells. “Suppressing” tumor cell growth means any or all of the following states: slowing, delaying, and stopping tumor growth, as well as tumor shrinkage.

“Delaying development” of a tumor means to defer, hinder, slow, retard, stabilize, and/or postpone development of the disease. This delay can be of varying lengths of time, depending on the history of the disease and/or individual being treated.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. Preferably, a vector of the invention comprises DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A polynucleotide or polynucleotide region has a certain percentage, for example at least 80, 85, 90, 91, 92, 93, 94, 95, 96, 97, 98, 99% or more sequence identity over its entire length when aligned, comparing the two sequences. The alignment may be carried out and the percent homology or sequence identity determined using software programs known in the art, for example those described in Current Protocols in Molecular Biology (F. M. Ausubel et al., eds., 1987) Supplement 30, section 7.7.18. A preferred alignment program is ALIGN Plus (Scientific and Educational Software, Pennsylvania), preferably using default parameters, which are as follows: mismatch=2; open gap=0; extend gap=2.

As used herein, a “transcriptional response element” or “transcriptional regulatory element”, or “TRE” is a polynucleotide sequence, preferably a DNA sequence, comprising one or more enhancer(s) and/or promoter(s) and/or promoter elements such as a transcriptional regulatory protein response sequence or sequences, which increases transcription of an operably linked polynucleotide in a host cell that allows a TRE to function.

As used herein, a FEN1 TRE is a cancer-specific transcriptional response element, which preferentially directs gene expression in cancer cells. A FEN1 TRE of the invention comprises a promoter and/or enhancer component of the 5′ sequence to a FEN1 gene. A FEN1 TRE may further comprise an additional enhancer and/or promoter element, which may or may not be derived from the FEN1 gene.

“Under transcriptional control” is a term well understood in the art and indicates that transcription of a polynucleotide sequence, usually a DNA sequence, depends on its being operably (operatively) linked to an element which contributes to the initiation of, or promotes, transcription.

The term “operably linked” relates to the orientation of polynucleotide elements in a functional relationship. A TRE is operably linked to a coding sequence if the TRE promotes transcription of the coding sequence. Operably linked means that the DNA sequences being linked are generally contiguous and, where necessary to join two protein coding regions, contiguous and in the same reading frame. However, since enhancers generally function when separated from the promoter by several kilobases and intronic sequences may be of variable length, some polynucleotide elements may be operably linked but not contiguous.

The term “vector”, as used herein, refers to a nucleic acid construct designed for transfer between different host cells. Vectors may be, for example, “cloning vectors” which are designed for isolation, propagation and replication of inserted nucleotides, “expression vectors” which are designed for expression of a nucleotide sequence in a host cell, a “viral vector” which is designed to result in the production of a recombinant virus or virus-like particle, or “shuttle vectors”, which comprise the attributes of more than one type of vector. Any vector for use in gene introduction can basically be used as a “vector” into which the DNA having TRE activity is introduced. Viral vectors, such as retrovirus vectors, adenovirus vectors, or adeno associated virus vectors, and non-viral vectors such as liposomes may be used. Plasmid vectors may also find use in practicing the present invention. The term vector as it applies to the present invention is used to describe a recombinant vector, e.g., a plasmid or viral vector (including a replication defective or replication competent viral vector) comprising a FEN1 TRE.

The terms “virus”, “viral particle”, “vector particle”, “viral vector particle”, and “virion” are used interchangeably and are to be understood broadly as meaning infectious viral particles that are formed when, e.g., a viral vector of the invention is transduced into an appropriate cell or cell line for the generation of infectious particles. Viral particles according to the invention may be utilized for the purpose of transferring nucleic acids (e.g. DNA or RNA) into cells either in vitro or in vivo.

The term “replication defective” as used herein relative to a viral vector of the invention means the viral vector cannot further replicate and package its genomes. For example, when the cells of a subject are infected with rAAV virions, the heterologous gene is expressed in the patient's cells, however, due to the fact that the patient's cells lack AAV rep and cap genes and the adenovirus accessory function genes, the rAAV is replication defective and wild-type AAV cannot be formed in the patient's cells.

As used herein, “packaging system” refers to a set of viral constructs comprising genes that encode viral proteins involved in packaging a recombinant virus. Typically, the constructs of the packaging system will ultimately be incorporated into a packaging cell.

The term “replication competent” as used herein may also be referred to as “replication conditional” relative to a viral vector of the invention. The term means the vector can selectively replicate in particular cell types (“target cells”), e.g., cancer cells and preferentially effect cytolysis of those cells. The term “replication-competent” as used herein relative to the viral vectors of the invention means the viral vectors and particles preferentially replicate in certain types of cells or tissues but to a lesser degree or not at all in other types. In one embodiment of the invention, the viral vector and/or particle selectively replicates in tumor cells and or abnormally proliferating tissue, such as solid tumors and other neoplasms. Such viruses may be referred to as “oncolytic viruses” or “oncolytic vectors” and may be considered to be “cytolytic” or “cytopathic” and to effect selective cytolysis” of target cells.

The term “plasmid” as used herein refers to a DNA molecule that is capable of autonomous replication within a host cell, either extrachromosomally or as part of the host cell chromosome(s). The starting plasmids herein are commercially available, are publicly available on an unrestricted basis, or can be constructed from such available plasmids as disclosed herein and/or in accordance with published procedures. In certain instances, as will be apparent to the ordinarily skilled artisan, other plasmids known in the art may be used interchangeably with plasmids described herein.

The terms “complement” and “complementary” refer to two nucleotide sequences that comprise antiparallel nucleotide sequences capable of pairing with one another upon formation of hydrogen bonds between the complementary base residues in the antiparallel nucleotide sequences.

The term “expression” refers to the transcription and/or translation of an endogenous gene, transgene or coding region in a cell.

By “transcriptional activation” or an “increase in transcription,” it is intended that transcription is increased above basal levels in a normal, i.e. non-transformed cell by at least about 2 fold, preferably at least about 5 fold, preferably at least about 10 fold, more preferably at least about 20 fold, more preferably at least about 50 fold, more preferably at least about 100 fold, more preferably at least about 200 fold, even more preferably at least about 400 fold to about 500 fold, even more preferably at least about 1000 fold. Basal levels are generally the level of activity (if any) in a non-target cell (i.e., a different cell type), or the level of activity (if any) of a reporter construct lacking a FEN1 TRE as tested in a target cell line. When the TRE controls a gene necessary for viral replication or expression of a gene, the replication of virus or expression of the gene, is significantly higher in the target cells, as compared to a control cell, usually at least about 2-fold higher, preferably, at least about 5-fold higher, more preferably, at least about 10-fold higher, still more preferably at least about 50-fold higher, even more preferably at least about 100-fold higher, still more preferably at least about 400- to 500-fold higher, still more preferably at least about 1000-fold higher, most preferably at least about 1×10⁶ higher. Most preferably, the TRE controls expression of a viral gene or transgene solely in the target cells (that is, does not replicate or replicates at a very low levels in non-target cells).

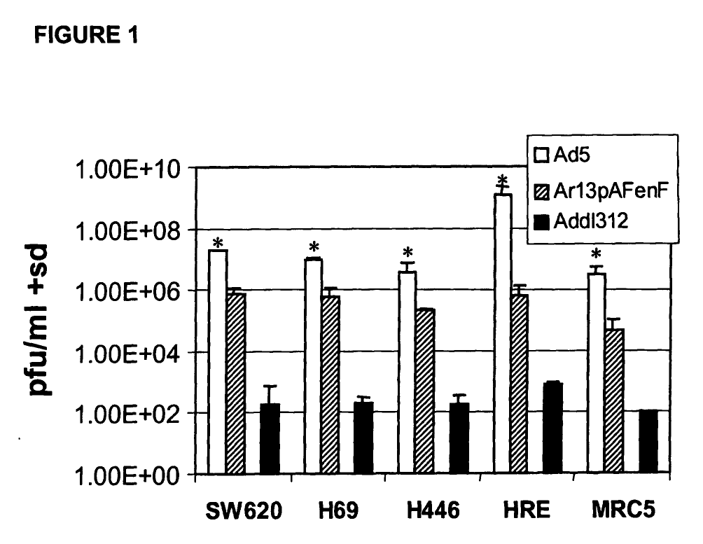

A “termination signal sequence” within the meaning of the invention may be any genetic element that causes RNA polymerase to terminate transcription, such as for example a polyadenylation signal sequence. A polyadenylation signal sequence is a recognition region necessary for endonuclease cleavage of an RNA transcript that is followed by the polyadenylation consensus sequence AATAAA. A polyadenylation signal sequence provides a “polyA site”, i.e. a site on a RNA transcript to which adenine residues will be added by post-transcriptional polyadenylation. Polyadenylation signal sequences are useful insulating sequences for transcription units within eukaryotic cells and eukaryotic viruses. Generally, the polyadenylation signal sequence includes a core poly(A) signal that consists of two recognition elements flanking a cleavage-polyadenylation site (e.g., FIG. 1 of WO 02/067861 and WO 02/068627). The choice of a suitable polyadenylation signal sequence will consider the strength of the polyadenylation signal sequence, as completion of polyadenylation process correlates with poly(A) site strength (Chao et al., Molecular and Cellular Biology, 1999, 19:5588-5600). For example, the strong SV40 late poly(A) site is committed to cleavage more rapidly than the weaker SV40 early poly(A) site. The person skilled in the art will consider choosing a stronger polyadenylation signal sequence if a more substantive reduction of nonspecific transcription is required in a particular vector construct. In principle, any polyadenylation signal sequence may be useful for the purposes of the present invention. However, in some embodiments of this invention the termination signal sequence is either the SV40 late polyadenylation signal sequence or the SV40 early polyadenylation signal sequence. Usually, the termination signal sequence is isolated from its genetic source and inserted into a vector of the invention at a suitable position upstream of a FEN1 TRE.

The term “enhancer” within the meaning of the invention may be any genetic element, e.g., a nucleotide sequence, that increases transcription of a coding sequence operatively linked to a promoter to an extent greater than the transcription activation effected by the promoter itself when operatively linked to the coding sequence, i.e. it increases transcription from the promoter in certain cells or even all cells.

A “multicistronic transcript” refers to a mRNA molecule that contains more than one protein coding region, or cistron. An mRNA comprising two coding regions is denoted a “bicistronic transcript.” The “5′-proximal” coding region or cistron is the coding region whose translation initiation codon (usually AUG) is closest to the 5′-end of a multicistronic mRNA molecule. A “5′-distal” coding region or cistron is one whose translation initiation codon (usually AUG) is not the closest initiation codon to the 5′ end of the mRNA. The terms “5′-distal” and “downstream” are used synonymously to refer to coding regions that are not adjacent to the 5′ end of a mRNA molecule.

As used herein, “co-transcribed” means that two (or more) coding regions of polynucleotides are under transcriptional control of a single transcriptional control or regulatory element.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson R J. Howell M T, Kaminski A (1990) Trends Biochem Sci 15(12):477-83) and Jackson R J and Kaminski, A. (1995) RNA 1(10):985-1000. The present invention encompasses the use of any IRES element, which is able to promote direct internal ribosome entry to the initiation codon of a cistron. “Under translational control of an IRES” as used herein means that translation is associated with the IRES and proceeds in a cap-independent manner. Examples of “IRES” known in the art include, but are not limited to IRES obtainable from picornavirus (Jackson et al., 1990, Trends Biochem Sci 15(12):477483); and IRES obtainable from viral or cellular mRNA sources, such as for example, immunoglobulin heavy-chain binding protein (BiP), the vascular endothelial growth factor (VEGF) (Huez et al. (1998) Mol. Cell. Biol. 18(11):6178-6190), the fibroblast growth factor 2, and insulin-like growth factor, the translational initiation factor eIF4G, yeast transcription factors TFIID and HAP4. IRES have also been reported in different viruses such as cardiovirus, rhinovirus, aphthovirus, HCV, Friend murine leukemia virus (FrMLV) and Moloney murine leukemia virus (MoMLV). As used herein, “IRES” encompasses functional variations of IRES sequences as long as the variation is able to promote direct internal ribosome entry to the initiation codon of a cistron. In preferred embodiments, the IRES is mammalian. In other embodiments, the IRES is viral or protozoan. In one illustrative embodiment disclosed herein, the IRES is obtainable from encephelomycarditis virus (ECMV) (commercially available from Novogen, Duke et al. (1992) J. Virol 66(3):1602-1609). In another illustrative embodiment disclosed herein, the IRES is from VEGF. Examples of IRES sequences are described in U.S. Pat. No. 6,692,736.

A “self-processing cleavage site” or “self-processing cleavage sequence” as referred to herein is a DNA or amino acid sequence, wherein upon translation, rapid intramolecular (cis) cleavage of a polypeptide comprising the self-processing cleavage site occurs to result in expression of discrete mature protein or polypeptide products. Such a “self-processing cleavage site”, may also be referred to as a post-translational or co-translational processing cleavage site, e.g., a 2A site, sequence or domain. A 2A site, sequence or domain demonstrates a translational effect by modifying the activity of the ribosome to promote hydrolysis of an ester linkage, thereby releasing the polypeptide from the translational complex in a manner that allows the synthesis of a discrete downstream translation product to proceed (Donnelly, 2001). Alternatively, a 2A site, sequence or domain demonstrates “auto-proteolysis” or “cleavage” by cleaving its own C-terminus in cis to produce primary cleavage products (Furler; Palmenberg, Ann. Rev. Microbiol. 44:603-623 (1990)).

As discussed herein, a FEN1 TRE can be of varying lengths, and of varying sequence composition. Embodiments of the invention include vectors comprising a FEN1 TRE, wherein the FEN1 TRE comprises a nucleotide sequence selected from the group consisting of: (a) the 2259 bp sequence shown in SEQ ID NO:1; (b) a fragment of the 2259 bp sequence shown in SEQ ID NO: 1, wherein the fragment has tumor selective transcriptional regulatory activity; (c) a nucleotide sequence having at least 80, 85, 90, 91, 92, 93, 94, 95, 96, 97, 98, 99% or more % identity over its entire length to the 2259 bp sequence shown in SEQ ID NO: 1 when compared and aligned for maximum correspondence, as measured using a standard sequence comparison algorithm (described below) or by visual inspection, wherein the nucleotide sequence has tumor selective transcriptional regulatory activity; and (d) a nucleotide sequence having a full-length complement that hybridizes under stringent conditions to the 2259 bp sequence shown in SEQ ID NO:1, wherein the nucleotide sequence tumor selective transcriptional regulatory activity. Preferably, the given % sequence identity exists over a region of the sequences that is at least about 50 nucleotides in length, more preferably over a region of at least about 100 nucleotides, and even more preferably over a region of at least about 200 nucleotides. Most preferably, the given % sequence identity exists over the entire length of the sequences.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), by the BLAST algorithm, Altschul et al., J Mol. Biol. 215: 403-410 (1990), with software that is publicly available through the National Center for Biotechnology Information (http://www.ncbi.nlm.nih.gov/), or by visual inspection (see generally, Ausubel et al., infra). For purposes of the present invention, optimal alignment of sequences for comparison is most preferably conducted by the local homology algorithm of Smith & Waterman, Adv. Appl. Math. 2: 482 (1981).

The terms “identical” or percent “identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the sequence comparison algorithms described herein, e.g. the Smith-Waterman algorithm, or by visual inspection.

In one embodiment, a FEN1 TRE according to the present invention has a full-length complement that hybridizes to the 2259 bp sequence shown in SEQ ID NO:1 under stringent conditions. The phrase “hybridizing to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent wash conditions” in the context of nucleic acid hybridization experiments such as Southern and Northern hybridizations are sequence dependent, and are different under different environmental parameters. Longer sequences hybridize at higher temperatures. An extensive guide to the hybridization of nucleic acids is found in Tijssen (1993) Laboratory Techniques in Biochemistry and Molecular Biology-Hybridization with Nucleic Acid Probes part 1 chapter 2 “Overview of principles of hybridization and the strategy of nucleic acid probe assays” Elsevier, New York. Generally, highly stringent hybridization and wash conditions are selected to be about 50° C. to 20° C. (preferably 5° C.) lower than the thermal melting point (Tm) for the specific sequence at a defined ionic strength and pH. Typically, under highly stringent conditions a probe will hybridize to its target subsequence, but to no other sequences.

The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleic acids that have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization.

As used herein, “transgene” refers to a polynucleotide that can be expressed, via recombinant techniques, in a non-native environment or heterologous cell under appropriate conditions. The transgene may be derived from the same type of cell in which it is to be expressed, but introduced from an exogenous source, modified as compared to a corresponding native form and/or expressed from a non-native site, or it may be derived from a heterologous cell. “Transgene” is synonymous with “exogenous gene”, “foreign gene” and “heterologous gene”. A transgene may be a therapeutic gene.

As used herein, a “therapeutic” gene refers to a transgene that, when expressed, confers a beneficial effect on the cell or tissue in which it is present, or on a mammal in which the gene is expressed. Examples of beneficial effects include amelioration of a sign or symptom of a condition or disease, prevention or inhibition of a condition or disease, or conferral of a desired characteristic. Therapeutic genes include genes that correct a genetic deficiency in a cell or mammal.

In the context of a vector for use in practicing the present invention, a “heterologous polynucleotide” or “heterologous gene” or “transgene” is any polynucleotide or gene that is not present in the corresponding wild-type vector or virus. Examples of preferred transgenes for inclusion in the vectors of the invention, are provided hereinbelow.

In the context of a vector for use in practicing the present invention, a “heterologous” promoter or enhancer is one which is not associated with or derived from the corresponding wild-type vector or virus.

In the context of a FEN1 TRE, a “heterologous” promoter or enhancer is one which is derived from a gene other than the FEN1 gene.

In the context of a vector for use in practicing the present invention, an “endogenous” promoter, enhancer or TRE is native to, or derived from the corresponding wild-type vector or virus.

“Replication” and “propagation” are used interchangeably and refer to the ability of a viral vector of the invention to reproduce or proliferate. These terms are well understood in the art. For purposes of this invention, replication involves production of virus proteins and is generally directed to reproduction of virus. Replication can be measured using assays standard in the art and described herein, such as a virus yield assay, burst assay or plaque assay. “Replication” and “propagation” include any activity directly or indirectly involved in the process of virus manufacture, including, but not limited to, viral gene expression; production of viral proteins, nucleic acids or other components; packaging of viral components into complete viruses and cell lysis.

“Preferential replication” and “selective replication” and “specific replication” may be used interchangeably and mean that the virus replicates more in a target cancer cell than in a non-cancer cell. Preferably, the virus replicates at a significantly higher rate in target cells than non target cells; preferably, at least about 3-fold higher, more preferably, usually at least about 10-fold higher, it may be at least about 50-fold higher, and in some instances at least about 100-fold, 400-fold, 500-fold, 1000-fold or even 1×10⁶ higher. In one embodiment, the virus replicates only in the target cells (that is, does not replicate at all or replicates at a very low level in non-target cells).

An “individual” is a vertebrate, preferably a mammal, more preferably a human. Mammals include, but are not limited to, farm animals, sport animals, rodents, primates, and pets. A “host cell” includes an individual cell or cell culture which can be or has been a recipient of a vector of this invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a vector of this invention.

As used herein, “cytotoxicity” is a term well understood in the art and refers to a state in which a cell's usual biochemical or biological activities are compromised (i.e., inhibited). These activities include, but are not limited to, metabolism; cellular replication; DNA replication; transcription; translation; uptake of molecules. “Cytotoxicity” includes cell death and/or cytolysis. Assays are known in the art which indicate cytotoxicity, such as dye exclusion, ³H-thymidine uptake, and plaque assays.

The terms “selective cytotoxicity” and “specific cytotoxicity” are used interchangeably and as used herein, refer to the cytotoxicity conferred by a vector of the invention on a cell which allows or induces a FEN1 TRE to function (referred to herein as a “target cell”) when compared to the cytotoxicity conferred by a vector of the present invention on a cell which does not allow a FEN1 TRE to function (a “non-target cell”). Such cytotoxicity may be measured, for example, by plaque assays, by reduction or stabilization in size of a tumor comprising target cells, or the reduction or stabilization of serum levels of a marker characteristic of the tumor cells, or a tissue-specific marker, e.g., a cancer marker.

The terms “candidate bioactive agent,” “drug candidate” “compound” or grammatical equivalents as used herein describes any molecule, e.g., protein, oligopeptide, small organic molecule, polysaccharide, polynucleotide, etc., to be tested for bioactive agents that are capable of directly or indirectly altering the cancer phenotype or the expression of a cancer sequence, including both nucleic acid sequences and protein sequences. In preferred embodiments, the bioactive agents modulate the expression profiles, or expression profile nucleic acids or proteins provided herein. In a particularly preferred embodiment, the candidate agent suppresses a cancer phenotype, for example to a normal tissue fingerprint. Similarly, the candidate agent preferably suppresses a severe cancer phenotype. Generally pluralities of assay mixtures are run in parallel with different agent concentrations to obtain a differential response to the various concentrations. Typically, one of these concentrations serves as a negative control, i.e., at zero concentration or below the level of detection.

FEN1 Transcriptional Response Elements of the Invention

A FEN1 TRE is a cancer-specific transcriptional response element, which preferentially directs gene expression in cancer cells. A FEN1 TRE of the invention comprises a promoter and/or enhancer component of the sequence 5′ to a FEN1 gene. The invention provides novel FEN1 regulatory sequences (set forth in SEQ ID NO:1), wherein the sequences provide for enhanced expression of an operably linked gene in cancer cells. This region of DNA contains the native transcriptional elements that direct expression of the FEN1 gene.

A FEN1 TRE of the present invention finds utility in vector-mediated delivery and in vivo expression of polynucleotides encoding proteins that are effective in the treatment of cancer.

In addition to the FEN1 TRE, a vector for use in practicing the invention may further comprise promoters and/or enhancers derived from the same or different genes. Such additional regulatory elements may be operably linked to a viral gene essential for replication or to a transgene.

A FEN1 TRE comprises a mammalian cancer-specific enhancer and/or promoter. Preferred FEN1 TREs comprise a FEN1 enhancer and/or promoter and are of human, rat or mouse origin, including promoter and enhancer elements and transcription factor binding sequences from the 5′ FEN1 sequence set forth in SEQ ID NO:1. The term “FEN1 promoter” refers to the native FEN1 promoter and functional fragments, mutations and derivatives thereof. A FEN1 TRE contains the native promoter elements that direct expression of an operably linked gene. Usually a promoter region will have at least about 100 nt of sequence located 5′ to the gene and may further comprise, but not always, a TATA box and/or CAAT box motif sequence.

The FEN1 TRE does not have to include the full-length wild type promoter and/or enhancer. One skilled in the art knows how to derive fragments from a FEN1 TRE and test them for the desired specificity. A FEN1 promoter fragment of the present invention has promoter activity specific for tumor cells, i.e. drives tumor selective expression of an operatively linked coding sequence. In one embodiment, the FEN1 TRE of the invention is a mammalian FEN1 TRE and in another embodiment it is a human FEN1 (hFEN1) TRE. In another embodiment of the invention, the FEN1 TRE consists essentially of SEQ ID NO:1, which is a 2259 bp fragment of the wild type hFEN1 TRE.

The sequence of this 5′ region, and further 5′ upstream sequences may be utilized to direct gene expression, including enhancer binding sites, that provide for expression in tissues where FEN1 is expressed, e.g. carcinoma cells. Sequence alterations, including substitutions, deletions and additions, may be introduced into the TRE region to determine the effect of altering expression in experimentally defined systems. Methods for the identification of specific DNA motifs involved in the binding of transcriptional factors are known in the art, e.g. sequence similarity to known binding motifs, gel retardation studies, etc.

FEN1 regulatory sequences may be used to identify cis acting sequences required for transcriptional or translational regulation of FEN1 expression, i.e., in different stages of metastasis, and to identify cis acting sequences and trans acting factors that regulate or mediate expression. Such transcription or translational control regions may be operably linked to a gene of interest in order to promote expression of a protein of interest in cultured cells, or in embryonic, fetal or adult tissues, and for gene therapy.

A FEN1 TRE can also comprise multimers. For example, a FEN1 TRE can comprise a tandem series of at least two, at least three, at least four, or at least five promoter fragments. Alternatively, a FEN1 TRE may have one or more promoter regions along with one or more enhancer regions. These multimers may also contain a heterologous promoter and/or enhancer sequences and/or transcription factor binding sites.

The promoter enhancer and/or transcription factor binding site components of a FEN1 TRE may be in any orientation and/or distance from the coding sequence of interest, as long as the desired target cell-specific transcriptional activity is obtained. Transcriptional activation can be measured in a number of ways known in the art, but is generally measured by detection and/or quantitation of mRNA or the protein product of the coding sequence under control of (i.e., operably linked to) the FEN1 TRE.

The term “composite TRE” refers to a TRE that comprises transcriptional regulatory elements that are not naturally found together, usually providing a non-native combination of promoters and enhancer, for example, a heterologous combination of promoter and enhancer and/or transcription factor binding sites; a combination of human and mouse promoter and enhancer; two or more enhancers in combination with a promoter; multimers of the foregoing; and the like. At least one of the promoter, enhancer or and/or transcription factor binding site elements will be cancer specific, for example the FEN1 promoter in combination with an enhancer. In other embodiments, two or more of the elements will provide cancer specificity. A composite TRE comprising regulatory elements from two or more sources may be used to regulate one or more genes.

A TRE for use in the present vectors may or may not be inducible. As is known in the art, the activity of TREs can be inducible. Inducible TREs generally exhibit low activity in the absence of inducer, and are up-regulated in the presence of inducer. Inducers include, for example, nucleic acids, polypeptides, small molecules, organic compounds and/or environmental conditions such as temperature, pressure or hypoxia. Inducible TREs may be preferred when expression is desired only at certain times or at certain locations, or when it is desirable to titrate the level of expression using an inducing agent.

A TRE for use in the present vectors may or may not comprise a silencer. The presence of a silencer (i.e., a negative regulatory element known in the art) can assist in shutting off transcription (and thus replication) in non-target cells. Thus, the presence of a silencer can confer enhanced cell-specific vector replication by more effectively preventing replication in non-target cells. Alternatively, the lack of a silencer may stimulate replication in target cells, thus conferring enhanced target cell-specificity.

A “functionally-preserved variant” of a FEN1 TRE differs, usually in sequence, but still retains the biological activity, e.g., cancer cell-specific transcriptional activity of the corresponding native or parent FEN1 TRE, although the degree of activation may be altered. The difference in sequence may arise from, for example, single base mutation(s), addition(s), deletion(s), and/or modification(s) of the bases. The difference can also arise from changes in the sugar(s), and/or linkage(s) between the bases of a FEN1 TRE. For example, certain point mutations within sequences of TREs have been shown to decrease transcription factor binding and stimulation of transcription (see Blackwood, et al. (1998) Science 281:60-63, and Smith et al. (1997) J. Biol. Chem. 272:27493-27496). Certain mutations are also capable of increasing TRE activity. Testing the effect of altered bases may be performed in vitro or in vivo by any method known in the art, such as mobility shift assays, or transfecting vectors containing these alterations in TRE functional and TRE non-functional cells. Additionally, one of skill in the art would recognize that point mutations and deletions can be made to a TRE sequence without altering the ability of the sequence to regulate transcription. It will be appreciated that typically a “functionally-preserved variant” of a FEN1 TRE will hybridize to the parent sequence under conditions of high stringency. Exemplary high stringency conditions include hybridization at about 65° C. in about 5×SSPE and washing at about 65° C. in about 0.1×SSPE (where 1×SSPE=0.15 sodium chloride, 0.010 M sodium phosphate, and 0.001 M disodium EDTA). Further examples of high stringency conditions are provided in: Maniatis, et al., MOLECULAR CLONING: A LABORATORY MANUAL, 2d Edition (1989); and Ausubel, F. M., et al., Eds., CURRENT PROTOCOLS IN MOLECULAR BIOLOGY, John Wiley & Sons, Inc., Copyright (c)1987, 1988, 1989,1990 by Current Protocols.

In some instances, a “functionally-preserved variant” of a FEN1 TRE is a fragment of a native or parent FEN1 TRE. The term “fragment,” when referring to a FEN1 TRE, refers to a sequence that is the same as part of, but not all of, the nucleic acid sequence of a native or parental FEN1 TRE. Such a fragment either exhibits essentially the same biological function or activity as the native or parental FEN1 TRE; for example, a fragment which retains the cancer cell-specific transcription activity of the corresponding native or parent FEN1 TRE, although the degree of activation may be altered.

Activity of a TRE can be determined, for example, as follows. A TRE polynucleotide sequence or set of such sequences can be generated using methods known in the art, such as chemical synthesis, site-directed mutagenesis, PCR, and/or recombinant methods. The sequence(s) to be tested can be inserted into a vector containing a promoter (if no promoter element is present in the TRE) and an appropriate reporter gene encoding a reporter protein, such as chloramphenicol acetyl transferase (CAT), β-galactosidase (encoded by the lacZ gene), luciferase (encoded by the luc gene), alkaline phosphatase (AP), green fluorescent protein (GFP), and horseradish peroxidase (HRP). Such vectors and assays are readily available, from, inter alia, commercial sources. Plasmids thus constructed are transfected into a suitable host cell to test for expression of the reporter gene as controlled by the putative TRE using transfection methods known in the art, such as calcium phosphate precipitation, electroporation, liposomes, DEAE dextran-mediated transfer, particle bombardment or direct injection. TRE activity is measured by detection and/or quantitation of reporter gene-derived mRNA and/or protein. The reporter protein product can be detected directly (e.g., immunochemically) or through its enzymatic activity, if any, using an appropriate substrate. Generally, to determine cell specific activity of a TRE, a TRE-reporter gene construct is introduced into a variety of cell types. The amount of TRE activity is determined in each cell type and compared to that of a reporter gene construct lacking the TRE. A TRE is determined to be cell-specific if it is preferentially functional in one cell type, compared to a different cell type.

Gene Transfer Vectors Of The Invention

The present invention contemplates the use of any of a variety of vectors for introduction of the vector or a transgene into mammalian cells relying on aFEN1 TRE of the invention to effect cancer specific expression of an operably linked gene. Exemplary vectors include but are not limited to, viral and non-viral vectors, such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV40) vectors, bovine papilloma virus vectors, Epstein-Barr virus, herpes virus vectors, vaccinia virus vectors, Harvey murine sarcoma virus vectors, murine mammary tumor virus vectors, Rous sarcoma virus vectors and nonviral plasmids. In one preferred approach, the vector is a viral vector. Viral vectors can efficiently transduce cells and introduce their own DNA into a host cell. In generating recombinant viral vectors, non-essential genes are typically replaced with a gene or coding sequence for a heterologous (or non-native) protein.

Methods that are well known to those skilled in the art can be used to construct expression vectors containing coding sequences and appropriate transcriptional and translational control signals, including a cancer specific control signal, for specific expression of an exogenous gene when introduced into a cell. These methods include, for example, in vitro recombinant DNA techniques, synthetic techniques, and in vivo genetic recombination. Alternatively, RNA capable of encoding gene product sequences may be chemically synthesized using, for example, synthesizers. See, for example, the techniques described in “Oligonucleotide Synthesis”, 1984, Gait, M. J. ed., IRL Press, Oxford. In constructing viral vectors, non-essential genes may be replaced with one or more genes encoding one or more therapeutic compounds or factors. Typically, the vector comprises an origin of replication and the vector may or may not also comprise a “marker” or “selectable marker” function by which the vector can be identified and selected. While any selectable marker can be used, selectable markers for use in such expression vectors are generally known in the art and the choice of the proper selectable marker will depend on the host cell. Examples of selectable marker genes which encode proteins that confer resistance to antibiotics or other toxins include ampicillin, methotrexate, tetracycline, neomycin (Southern et al., J., J Mol Appl Genet. 1982;1(4):32741 (1982)), mycophenolic acid (Mulligan et al., Science 209:1422-7 (1980)), puromycin, zeomycin, hygromycin (Sugden et al., Mol Cell Biol. 5(2):410-3 (1985)) or G418.

Reference to a vector or other DNA sequences as “recombinant” merely acknowledges the operable linkage of DNA sequences which are not typically operably linked as isolated from or found in nature. Regulatory (expression/control) sequences are operatively linked to a nucleic acid coding sequence when the expression/control sequences regulate the transcription and, as appropriate, translation of the nucleic acid sequence. Thus expression/control sequences can include transcriptional regulatory elements, e.g., promoters and enhancers; transcription terminators; a start codon (i.e., ATG) in front of the coding sequined; splicing signal for introns and stop codons, etc.

In cases where an entire gene, including its own initiation codon and adjacent sequences, is inserted into the appropriate expression vector, no additional translational control signals may be needed. However, in cases where only a portion of the gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et at., 1987, Methods in Enzymol. 153:516-544). In some embodiments, specificity is conferred by preferential replication of the vector in target cells due to the FEN1 TRE driving transcription of a gene essential for replication. In other embodiments, efficacy is conferred by preferential transcription and/or translation of a transgene due to operable linakge to a FEN1 TRE.

In other words, the present invention relies upon the cancer-specific expression of a coding sequence operatively linked to a FEN1 TRE and the use of vectors comprising a FEN1 TRE as a means for targeting cancer cells. Such targeting may relate to replication of the vector and/or expression of a transgene encoded therein.

In one embodiment of a recombinant viral vector of the invention, the FEN1 TRE comprises a nucleotide sequence selected from the group consisting of: (a) the 2259 bp sequence shown in SEQ ID NO:1; (b) a fragment of the 2259 bp sequence shown in SEQ ID NO: 1, wherein the fragment has tumor selective transcriptional regulatory activity; (c) a nucleotide sequence having at least 80, 85, 90, 91, 92, 93, 94, 95, 96, 97, 98, 99% or more % identity over its entire length to the 2259 bp sequence shown in SEQ ID NO: 1 when compared and aligned for maximum correspondence, as measured using one of the sequence comparison algorithms described herein or by visual inspection, wherein the nucleotide sequence has tumor selective transcriptional regulatory activity; and (d) a nucleotide sequence having a full-length complement that hybridizes under stringent conditions to the 2259 bp sequence shown in SEQ ID NO:1, wherein the nucleotide sequence tumor selective transcriptional regulatory activity. Preferably, the given % sequence identity exists over a region of the sequences that is at least about 50 nucleotides in length, more preferably over a region of at least about 100 nucleotides, and even more preferably over a region of at least about 200 nucleotides. Most preferably, the given % sequence identity exists over the entire length of the sequences. In another embodiment of a recombinant viral vector of the invention, the FEN1 TRE sequence consists essentially of SEQ ID NO:1.

Functionally preserved variants of a FEN1 TRE sequence can be used in the vectors disclosed herein. Variant FEN1 TREs retain function in the target cell but need not exhibit maximal function. In fact, maximal transcriptional activation activity of a FEN1 TRE may not always be necessary to achieve a desired result, and the level of induction afforded by a fragment of a FEN1 TRE may be sufficient for certain applications. For example, if used for treatment or palliation of a disease state, less-than-maximal responsiveness may be sufficient if, for example, the target cells are not especially virulent and/or the extent of disease is relatively confined.

As discussed herein, a FEN1 TRE can be of varying lengths, and of varying sequence composition. The size of a FEN1 TRE is determined in part by the capacity of the viral vector, which in turn depends upon the contemplated form of the vector. Generally minimal sizes are preferred for FEN1 TREs, as this provides potential room for insertion of other sequences which may be desirable, such as transgenes, and/or additional regulatory sequences. In a preferred embodiment, such an additional regulatory sequence is an IRES or a self-processing cleavage sequence, such as a 2A sequence. However, if no additional sequences are contemplated, or if, for example the vector is a viral vector which will be maintained and delivered free of any viral packaging constraints, larger TRE sequences can be used as long as the resultant viral vector remains replication-competent.

A vector for use in practicing the invention may have co-transcribed first and second genes under control of a cancer, i.e. a colon cancer-specific TRE and the second gene may be under translational control of an internal ribosome entry site (IRES) or a self-processing cleavage sequence, such as a 2A sequence.

To minimize non-specific replication, endogenous viral TREs are preferably removed from the vector. Besides facilitating target cell-specific replication, removal of endogenous TREs also provides greater insert capacity in a vector, which may be of special concern if an adenoviral vector is used in order for the vector to be packaged within a virus particle. Even more importantly, deletion of endogenous TREs prevents the possibility of a recombination event whereby a heterologous TRE is deleted and the endogenous TRE assumes transcriptional control of its respective virus coding sequences. However, endogenous TREs can be maintained in the vector(s), provided that sufficient cell-specific replication preference is preserved. These embodiments are constructed by inserting heterologous TREs between an endogenous TRE and a replication gene coding segment. Requisite cancer-specific replication preference is determined by conducting assays that compare replication of the vector in a cell which allows function of the heterologous FEN1 TREs with replication in a cell which does not.

In another aspect, methods are provided for conferring selective cytotoxicity in target cancer cells by contacting the cells with a viral vector of the invention, whereby the vector enters the cell and propagates. The replication of viral vectors comprising a FEN1 TRE in cancer cells, as compared to non-cancer cells, or to normal, i.e. non-transformed cells, is at least about 3 fold greater and is usually about 10 fold greater, and may be about 100 fold greater, and in some instances is as much as about 1000 fold or more greater. The administration of virus may be combined with additional treatment(s) appropriate to the particular disease, e.g. antiviral therapy, chemotherapy, surgery, radiation therapy or immunotherapy. In some embodiments, this treatment suppresses tumor growth, e.g. by killing tumor cells. In other embodiments, the size and/or extent of a tumor is reduced, or its development delayed. Cytotoxicity is a term well understood in the art and refers to a state in which a cell's usual biochemical or biological activities are compromised (i.e., inhibited), including cell death and/or cytolysis. These activities include, but are not limited to, metabolism; cellular replication; DNA replication; transcription; translation; uptake of molecules. Assays known in the art as indicators of cytotoxicity, include dye exclusion, ³H-thymidine uptake, and plaque assays.

Adenoviral Vectors

In one aspect, the invention provides an adenoviral vector comprising a FEN1 TRE. The adenoviral vector may be replication defective or replication competent. In the case of replication competent adenoviral vectors, the vector comprises an adenovirus gene essential for replication, e.g. an early gene, under the transcriptional control of a FEN1 TRE. By providing one or more cancer-specific TREs, such a replication competent adenoviral vector effects specific replication and corresponding cytotoxicity in cancer cells.

As used herein, the terms “adenovirus” and “adenoviral particle” are used to include any and all viruses that may be categorized as an adenovirus, including any adenovirus that infects a human or an animal, including all groups, subgroups, and serotypes (see Table 1). Thus, as used herein, “adenovirus” and “adenovirus particle” refer to the virus itself or derivatives thereof and cover all serotypes and subtypes and both naturally occurring and recombinant forms, except where indicated otherwise. Such adenoviruses may be wildtype or may be modified in various ways known in the art or as disclosed herein. Such modifications include modifications to the adenovirus genome that is packaged in the particle in order to make an infectious virus. Such modifications include deletions known in the art, such as deletions in one or more of the E1a, E1b, E2a, E2b, E3, or E4 coding regions. The terms also include replication-specific adenoviruses; that is, viruses that preferentially replicate in certain types of cells or tissues but to a lesser degree or not at all in other types. Such viruses are sometimes referred to as “cytolytic” or “cytopathic” viruses (or vectors), and, if they have such an effect on neoplastic cells, are referred to as “oncolytic” viruses (or vectors).

A “replication competent adenovirus vector” or “replication competent adenoviral vector” (used interchangeably) of the invention is a polynucleotide construct, which exhibits preferential replication in primary cancer cells and contains a FEN1 TRE linked to an adenoviral gene. In some embodiments, an adenoviral vector of the invention includes a transgene, e.g., a therapeutic gene such as a cytokine gene. Exemplary adenoviral vectors of the invention include, but are not limited to, DNA, DNA encapsulated in an adenovirus coat, adenoviral DNA packaged in another viral or viral-like form (such as herpes simplex, and AAV), adenoviral DNA encapsulated in liposomes, adenoviral DNA complexed with polylysine, adenoviral DNA complexed with synthetic polycationic molecules, conjugated with transferrin, or complexed with a compound such as PEG to immunologically “mask” the antigenicity and/or increase half-life, or conjugated to a nonviral protein.

The adenoviral vector comprising a FEN1 TRE may further comprise one or more regulatory sequences, e.g. enhancers, promoters, transcription factor binding sites and the like, which may be derived from the same or different genes. The adenovirus vector may comprise co-transcribed first and second genes under control of a FEN1 TRE, wherein the second gene may be under translational control of an internal ribosome entry site (IRES) or a self-processing cleavage sequence, such as a 2A sequence. In some cases, the adenovirus vectors comprise more than two co-transcribed genes under control of a FEN1 TRE. The adenovirus vectors of the invention may or may not comprise the adenoviral E3 gene, an E3 sequence, or a portion thereof.

In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E3) will result in a recombinant virus that is viable and capable of expressing the gene product in infected hosts (see Logan & Shenk, 1984, Proc. Natl. Acad. Sci. USA 81:3655-3659). Standard systems for generating adenoviral vectors for expression of inserted sequences are available from commercial sources, for example the Adeno-X™ expression system from Clontech (Clontechniques (January 2000) p. 10-12).

In one preferred aspect, the adenoviral vectors described herein are replication-competent cancer-specific adenoviral vectors comprising an adenovirus gene, preferably a gene essential for replication under transcriptional control of a FEN1 TRE. In general, the adenoviral gene essential for replication is an early gene, e.g. one or more of E1A, E1B and E4. In some embodiments, an adenovirus vector is a replication competent cancer-specific vector comprising E1B, wherein E1B has a deletion of part or all of the 19-kDa region.

In another preferred aspect, the adenoviral vectors described herein are replication-competent cancer-specific adenoviral vectors comprising an adenovirus gene, preferably a gene essential for replication under transcriptional control of a FEN1 TRE. The vector further comprises one or more additional TREs, which may or may not be cancer-specific, e.g., colon cancer specific. The one or more additional TREs may be operably linked to an adenoviral gene essential for replication or a transgene, i.e., a therapeutic gene. In one aspect of the invention, the he one or more additional TREs is a colon cancer specific regulatory sequence such as a “plasminogen activator urokinase (uPA)” TRE, (“uPA-TRE”), described for example in WO98/39464 or a PRL-3 transcriptional regulatory element (“PRL-3-TRE”), described for example in WO 20004/009790. In a related aspect, the one or more additional TREs comprises a cell status TRE such as an “E2F promoter” or a “telomerase promoter” or “TERT promoter.

The protein urokinase plasminogen activator (uPA) and its cell surface receptor, urokinase plasminogen activator receptor (uPAR), are expressed in many of the most frequently-occurring neoplasms and appear to represent important proteins in cancer metastasis. Both proteins are implicated in breast, colon, prostate, liver, renal, lung and ovarian cancer. Sequence elements that regulate uPA and uPAR transcription have been extensively studied. Riccio et al. (1985) Nucleic Acids Res. 13:2759-2771; Cannio et al. (1991) Nucleic Acids Res. 19:2303-2308. See also, WO 98/39464.

The PRL-3 protein tyrosine phosphatase gene has been recently found to be specifically expressed at a high level in metastatic colon cancers (Saha et al. (2001) Science 294:1343). Originally identified as a member of a group of up-regulated genes in a metastatic colon cancer library, identified by the serial analysis of gene expression (SAGE), the PRL-3 gene was confirmed to be elevated in only the metastases, not the primary cancer or pre-malignant adenomas. Replication competent adenoviral vectors comprising PRL-3 transcriptional regulatory sequences are described in WO 20004/009790. Exemplary sequences for use in the present invention are the sequences are presented as a 0.6 kb and 1 kb sequence upstream of the translational start codon for the PRL-3 gene (identified as SEQ ID NO:1 and SEQ ID NO:2 in WO 20004/009790).

The term “telomerase promoter” or “TERT promoter” as used herein refers to a native TERT promoter and functional fragments, mutations and derivatives thereof. The TERT promoter does not have to be the full-length or wild type promoter. One skilled in the art knows how to derive fragments from a TERT promoter and test them for the desired selectivity. A TERT promoter fragment for use in the present invention has promoter activity selective for tumor cells, i.e. drives tumor selective expression of an operatively linked coding sequence. In one embodiment, the TERT promoter of the invention is a mammalian TERT promoter. In another embodiment, the mammalian TERT promoter is a human TERT (hTERT) promoter. See, e.g., WO 98/14593 and WO 00/46355 for exemplary TERT promoters that find utility in the compositions and methods of the present invention.

The term “E2F promoter” as used herein refers to a native E2F promoter and functional fragments, mutations and derivatives thereof. The E2F promoter does not have to be the full-length or wild type promoter. One skilled in the art knows how to derive fragments from an E2F promoter and test them for the desired selectivity. An E2F promoter fragment of the present invention has promoter activity selective for tumor cells, i.e. drives tumor selective expression of an operatively linked coding sequence. The term “tumor selective promoter activity” as used herein means that the promoter activity of a promoter fragment of the present invention in tumor cells is higher than in non-tumor cell types. A number of examples of E2F promoters are known in the art. See, e.g., Parr et al. Nature Medicine 1997:3(10) 1145-1149, WO 021067861, US20010053352 and WO 98/13508.

A TERT promoter according to the present invention has the sequence shown in SEQ ID NO:3 or is a full-length complement that hybridizes to the sequence shown in SEQ ID NO:3 under stringent conditions. An E2F promoter according to the present invention has the sequence shown in SEQ ID NO:8 or is a full-length complement that hybridizes to the sequence shown in SEQ ID NO:8 under stringent conditions. The phrase “hybridizing to” refers to the binding, duplexing, or hybridizing of a molecule to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

The adenoviral E1B 19-kDa region refers to the genomic region of the adenovirus E1B gene encoding the E1B 19-kDa product. According to wild-type Ad5, the E1B 19-kDa region is a 261 bp region located between nucleotide 1714 and nucleotide 2244. The E1B 19-kDa region has been described in, for example, Rao et al., Proc. Natl. Acad. Sci. USA, 89:7742-7746. The present invention encompasses deletion of part or all of the E1B 19-kDa region as well as embodiments wherein the E1B 19-kDa region is mutated, as long as the deletion or mutation lessens or eliminates the inhibition of apoptosis associated with E1B-1 9kDa.

The adenovirus vectors of the invention replicate preferentially in carcinoma cells, which replication preference is indicated by comparing the level of replication (e.g., cell killing and/or titer) in carcinoma cells to the level of replication in non-cells, normal or control cells. Comparison of the adenovirus titer of a carcinoma cell to the titer of a TRE inactive cell type provides a key indication that the overall replication preference is enhanced due to the replication in target cells as well as depressed replication in non-target cells. This is especially useful in the metastatic cancer context, in which targeted cell killing is desirable. Runaway infection is prevented due to the cell-specific requirements for viral replication. Without wishing to be bound by any particular theory, production of adenovirus proteins can serve to activate and/or stimulate the immune system, either generally or specifically toward target cells producing adenoviral proteins which can be an important consideration in the cancer context, where individuals are often moderately to severely immunocompromised.

In one aspect of the present invention, the adenovirus vectors comprise an intergenic IRES element(s) which links the translation of two or more genes. Adenovirus vectors comprising an IRES are stable and in some embodiments provide better specificity than vectors not containing an IRES. Another advantage of an adenovirus vector comprising an intergenic IRES is that the use of an IRES rather than a second TRE may provide additional space in the vector for an additional gene(s) such as a therapeutic gene. Accordingly, in one aspect of the invention, the viral vectors disclosed herein typically comprise at least one IRES within a multicistronic transcript, wherein production of the multicistronic transcript is regulated by a heterologous, target cell-specific TRE.

For adenovirus vectors comprising a second gene under control of an IRES, it is preferred that the endogenous promoter of the gene under translational control of an IRES be deleted so that the endogenous promoter does not interfere with transcription of the second gene. It is preferred that the second gene be in frame with the IRES if the IRES contains an initiation codon. If an initiation codon, such as ATG, is present in the IRES, it is preferred that the initiation codon of the second gene is removed and that the IRES and the second gene are in frame. Alternatively, if the IRES does not contain an initiation codon or if the initiation codon is removed from the IRES, the initiation codon of the second gene is used. In one embodiment, the adenovirus vectors comprise the adenovirus essential genes, E1A and E1B genes, under the transcriptional control of a heterologous FEN1 TRE, and an IRES introduced between E1A and E1B. Thus, both E1A and E1B are under common transcriptional control, and translation of E1B coding region is obtained by virtue of the presence of the IRES. In one embodiment, E1A has its endogenous promoter deleted.

In another embodiment, E1A has an endogenous enhancer deleted and in yet an additional embodiment, E1A has its endogenous promoter deleted and E1A enhancer deleted. In another embodiment, E1B has its endogenous promoter deleted. In yet further embodiments, E1B has a deletion of part or all of the 19-kDa region of E1B.

In one embodiment of a recombinant viral vector of the invention, the FEN1 TRE is a human FEN1 TRE. The results provided herein in Examples 1 and 2 show that the FEN1 gene is preferentially expressed in cancer cells.

In a preferred embodiment of a recombinant viral vector of the invention, the coding sequence of a gene essential for replication is selected from the group consisting of E1a, E1b, E2a, E2b and E4 coding sequences. In one embodiment, the FEN1 TRE is operatively linked to one of either the E1a, E1b or E4 coding sequence. In another embodiment, the vector further comprises an additional heterologous TRE, e.g., a uPA-TRE, PRL-3-TRE, hTERT TRE or E2F TRE, operatively linked to an E1a, E1b or E4 coding sequence. In one embodiment, the FEN1 TRE is operatively linked to the E1a coding sequence and a different TRE is operatively linked to the E1b or E4 coding sequence.

In another embodiment of a recombinant viral vector of the invention, the nucleic acid backbone further comprises a termination signal sequence upstream of the FEN1 TRE operatively linked to the coding sequence of a gene essential for replication of the recombinant viral vector. In one embodiment, the termination signal sequence is the SV40 early polyadenylation signal sequence. In another embodiment, the vector further comprises a deletion upstream of the termination signal sequence. For example, the vector may comprise a deletion between nucleotides corresponding to nucleotides 103 and 551 of the adenoviral type 5 backbone. Vectors based on other adenovirus serotypes may have the same corresponding nucleotides deleted.

An adenovirus vector may further include an additional heterologous TRE, which may or may not be operably linked to the same gene(s) as the target cell-specific TRE. For example a TRE (such as a cell type-specific or cell status-specific TRE) may be juxtaposed to a second type of target-cell-specific TRE. “Juxtaposed” means a target cell-specific TRE and a second TRE transcriptionally control the same gene. For these embodiments, the target cell-specific TRE and the second TRE may be in any of a number of configurations, including, but not limited to, (a) next to each other (i.e., abutting); (b) both 5′ to the gene that is transcriptionally controlled (i.e., may have intervening sequences between them); (c) one TRE 5′ and the other TRE 3′ to the gene.

In one embodiment, the adenoviral vector comprises a transgene which is inserted in the E3 region of the adenoviral nucleic acid backbone. For example, transgene may be inserted in place of the 19 kD or 14.7 kD E3 gene. In one aspect of this embodiment, the transgene encodes an immunostimulatory protein. In another aspect, the immunostimulatory protein is a cytokine such as GM-CSF. In yet another aspect, the transgene encodes an anti-angiogenic protein. In still another aspect, the transgene is a suicide gene.

The invention further provides a recombinant adenovirus particle comprising a recombinant adenoviral vector according to the invention. In one embodiment, a capsid protein of the adenovirus particle comprises a targeting ligand. In another embodiment, the capsid protein is a fiber protein. In one aspect, the capsid protein is a fiber protein and the ligand is in the HI loop of the fiber protein. The adenoviral vector particle may also include other mutations to the fiber protein. Examples of these mutations include, but are not limited to those described in US application no. 20040002060, WO 98/07877, WO 01/92299, and US Pat. Nos. 5,962,311, 6,153,435, and 6,455,314. These include, but are not limited to, mutations that decrease binding of the viral vector particle to a particular cell type or more than one cell type, enhance the binding of the viral vector particle to a particular cell type or more than one cell type and/or reduce the immune response to the adenoviral vector particle in an animal. In addition, the adenoviral vector particles of the present invention may also contain mutations to other viral capsid proteins. Examples of these mutations include, but are not limited to those described in U.S. Pat. Nos. 5,731,190, 6,127,525, and 5,922,315. Other mutated adenoviruses are described in U.S. Pat. Nos. 6,057,155, 5,543,328 and 5,756,086.

The adenovirus vectors of this invention can be prepared using recombinant techniques that are standard in the art. Generally, a FEN1 TRE is inserted 5′ to the adenoviral gene of interest, e.g. an adenoviral replication gene, including one or more early replication genes (although late gene(s) can be used). A FEN1 TRE can be prepared using oligonucleotide synthesis (if the sequence is known) or recombinant methods (such as PCR and/or restriction enzymes). Convenient restriction sites, either in the natural adeno-DNA sequence or introduced by methods such as PCR or site-directed mutagenesis, provide an insertion site for a FEN1 TRE. Accordingly, convenient restriction sites for annealing (i.e., inserting) a FEN1 TRE can be engineered onto the 5′ and 3′ ends of a FEN1 TRE using standard recombinant methods, such as PCR. In one embodiment, the TRE replaces at least one native adenovirus TRE.

Adenoviral vectors containing at least one gene essential for replication (e.g., E1a) under transcriptional control of a FEN1 TRE, are conveniently prepared by homologous recombination or in vitro ligation of two plasmids, one providing the left-hand portion of adenovirus and the other plasmid providing the right-hand region, one or more of which contains at least one adenovirus gene under control of a FEN1 TRE. If homologous recombination is used, the two plasmids should share at least about 500 bp of sequence overlap, although smaller regions of overlap will recombine, but usually with lower efficiencies. Each plasmid, as desired, may be independently manipulated, followed by cotransfection in a competent host, providing complementing genes as appropriate, or the appropriate transcription factors for initiation of transcription from a FEN1 TRE for propagation of the adenovirus. Plasmids are generally introduced into a suitable host cell (e.g. 293, PerC.6, Hela-S3 cells) using appropriate means of transduction, such as cationic liposomes or calcium phosphate. Alternatively, in vitro ligation of the right and left-hand portions of the adenovirus genome can also be used to construct recombinant adenovirus derivative containing all the replication-essential portions of adenovirus genome. Berkner et al. (1983) Nucleic Acid Research 11: 6003-6020; Bridge et al. (1989) J. Virol. 63: 631-638.

For convenience, plasmids are available that provide the necessary portions of adenovirus. Plasmid pXC.1 (McKinnon (1982) Gene 19:33-42) contains the wild-type left-hand end of Ad5. pBHG10 (Bett et al. (1994); Microbix Biosystems Inc., Toronto) provides the right-hand end of Ad5, with a deletion in E3. The deletion in E3 provides room in the virus to insert up to about a 3 KB TRE without deleting the endogenous enhancer/promoter. The gene for E3 is located on the opposite strand from E4 (r-strand). pBHG 11 provides an even larger E3 deletion (an additional 0.3 kb is deleted). Bett et al. (1994). Alternatively, the use of pBHGE3 (Microbix Biosystems, Inc.) provides the right hand end of Ad5, with a full-length of E3.

For manipulation of the early genes, the transcription start site of Ad5 E1A is at 498 and the ATG start site of the E1A coding segment is at 560 in the virus genome. This region can be used for insertion of a FEN1 TRE. A restriction site may be introduced by employing polymerase chain reaction (PCR), where the primer that is employed may be limited to the Ad5 genome, or may involve a portion of the plasmid carrying the Ad5 genomic DNA. For example, where pBR322 is used, the primers may use the EcoRI site in the pBR322 backbone and the Xbal site at nt 1339 of Ad5. By carrying out the PCR in two steps, where overlapping primers at the center of the region introduce a nucleotide sequence change resulting in a unique restriction site, one can provide for insertion of a FEN1 TRE at that site.

A similar strategy may also be used for insertion of a FEN1 TRE element in operative linkage to E1B. The E1B promoter of Ad5 consists of a single high-affinity recognition site for Spl and a TATA box. This region extends from Ad5 nt 1636 to 1701. By insertion of a TRE in this region, one can provide for cell-specific transcription of the E1B gene. By employing the left-hand region modified with the cell-specific response element regulating E1A, as the template for introducing a FEN1 TRE to regulate E1B, the resulting adenovirus vector will be dependent upon the cell-specific transcription factors for expression of both E1A and E1B. In some embodiments, part or all of the 19-kDa region of E1B is deleted.

Similarly, a FEN1 TRE can be inserted upstream of the E2 gene to make its expression cell-specific. The E2 early promoter, mapping in Ad5 from 27050-27150, consists of a major and a minor transcription initiation site, the latter accounting for about 5% of the E2 transcripts, two non-canonical TATA boxes, two E2F transcription factor binding sites and an ATF transcription factor binding site (for a detailed review of the E2 promoter architecture see Swaminathan et al., Curr. Topics in Micro. and Immunol. (1995) 199(part 3):177-194.

The E2 late promoter overlaps with the coding sequences of a gene encoded by the counterstrand and is therefore not amenable for genetic manipulation. However, the E2 early promoter overlaps only for a few base pairs with sequences coding for a 33 kD protein on the counterstrand. Notably, the Spel restriction site (Ad5 position 27082) is part of the stop codon for the above mentioned 33 kD protein and conveniently separates the major E2 early transcription initiation site and TATA-binding protein site from the upstream transcription factor binding sites E2F and ATF. Therefore, insertion of a FEN1 TRE having Spel ends into the Spel site in the 1-strand would disrupt the endogenous E2 early promoter of Ad5 and should allow cell-restricted expression of E2 transcripts.

For E4, one must use the right hand portion of the adenovirus genome. The E4 transcription start site is predominantly at about nt 35605, the TATA box at about nt 35631 and the first AUG/CUG of ORF I is at about nt 35532. Virtanen et al. (1984) J. Virol. 51: 822-831. Using any of the above strategies for the other genes, a FEN1 TRE may be introduced upstream from the transcription start site. For the construction of full-length adenovirus with a FEN1 TRE inserted in the E4 region, the co-transfection and homologous recombination are performed in W162 cells (Weinberg et al. (1983) Proc. Natl. Acad. Sci. 80:5383-5386) which provide E4 proteins in trans to complement defects in synthesis of these proteins.

An “E3 region” (used interchangeably with “E3”) is a term well understood in the art and means the region of the adenoviral genome that encodes the E3 gene products. Generally, the E3 region is located between about nucleotides 28583 and 30470 of the adenoviral genome. The E3 region has been described in various publications, including, for example, Wold et al. (1995) Curr. Topics Microbiol. Immunol. 199:237-274. A “portion” of the E3 region means less than the entire E3 region, and as such includes polynucleotide deletions as well as polynucleotides encoding one or more polypeptide products of the E3 region.

Adenoviral constructs containing an E3 region can be generated wherein homologous recombination between an E3-containing adenoviral plasmid, for example, BHGE3 (Microbix Biosystems Inc., Toronto) and a non-E3-containing adenoviral plasmid, is carried out.

Alternatively, an adenoviral vector comprising an E3 region can be introduced into cells, for example 293 cells, along with an adenoviral construct or an adenoviral plasmid construct, where they can undergo homologous recombination to yield adenovirus containing an E3 region. In this case, the E3-containing adenoviral vector and the adenoviral construct or plasmid construct contain complementary regions of adenovirus, for example, one contains the left-hand and the other contains the right-hand region, with sufficient sequence overlap as to allow homologous recombination.

Alternatively, an E3-containing adenoviral vector of the invention can be constructed using other conventional methods including standard recombinant methods (e.g., using restriction nucleases and/or PCR), chemical synthesis, or a combination of any of these. Further, deletions of portions of the E3 region can be created using standard techniques of molecular biology.

Insertion of an IRES into a vector is accomplished by methods and techniques that are known in the art and described herein supra, including but not limited to, restriction enzyme digestion, ligation, and PCR. A DNA copy of an IRES can be obtained by chemical synthesis, or by making a cDNA copy of, for example, a picornavirus IRES. See, for example, Duke et al. (1995) J. Virol. 66(3):1602-9) for a description of the EMCV IRES and Huez et al. (1998), Mol. Cell. Biol. 18(11):6178-90) for a description of the VEGF IRES. The internal translation initiation sequence is inserted into a vector genome at a site such that it lies upstream of a 5′-distal coding region in a multicistronic mRNA. For example, in a preferred embodiment of an adenovirus vector in which production of a bicistronic E1A-E1B mRNA is under the control of a FEN1 TRE, the E1B promoter is deleted or inactivated, and an IRES sequence is placed between E1A and E1B. In other embodiments, part or all of the 19-kDa region of E1B is deleted. IRES sequences of cardioviruses and certain aphthoviruses contain an AUG codon at the 3′ end of the IRES that serves as both a ribosome entry site and as a translation initiation site. Accordingly, this type of IRES is introduced into a vector so as to replace the translation initiation codon of the protein whose translation it regulates. However, in an IRES of the entero/rhinovirus class, the AUG at the 3′ end of the IRES is used for ribosome entry only, and translation is initiated at the next downstream AUG codon. Accordingly, if an entero/rhinovirus IRES is used in a vector for translational regulation of a downstream coding region, the AUG (or other translation initiation codon) of the downstream gene is retained in the vector construct.

In some embodiments, the adenovirus death protein (ADP), encoded within the E3 region, is maintained in an adenovirus vector. The ADP gene, under control of the major late promoter (MLP), appears to code for a protein (ADP) that is important in expediting host cell lysis. Tollefson et al. (1996) J. Virol. 70(4):2296; Tollefson et al. (1992) J. Virol. 66(6):3633. Thus, adenoviral vectors containing the ADP gene may render the adenoviral vector more potent, making possible more effective treatment and/or a lower dosage requirement.

Accordingly, in one embodiment the invention provides adenovirus vectors in which an adenovirus gene is under transcriptional control of a first transactivator regulated transcriptional regulatory element and a polynucleotide sequence encoding an ADP under control of a second transactivator regulated transcriptional regulatory element, and wherein preferably the adenovirus gene is essential for replication. The DNA sequence encoding ADP and the amino acid sequence of an ADP are publicly available. Briefly, an ADP coding sequence is obtained preferably from Ad2 (since this is the strain in which ADP has been more fully characterized) using techniques known in the art, such as PCR. Preferably, the Y leader (which is an important sequence for correct expression of late genes) is also obtained and ligated to the ADP coding sequence. The ADP coding sequence (with or without the Y leader) can then be introduced into the adenoviral genome, for example, in the E3 region (where the ADP coding sequence will be driven by the MLP). The ADP coding sequence could also be inserted in other locations of the adenovirus genome, such as the E4 region. Alternatively, the ADP coding sequence could be operably linked to a different type of TRE, including, but not limited to, another viral TRE.

Methods of packaging polynucleotides into adenovirus particles are known in the art and are also described in co-owned PCT PCT/US98/04080. The preferred packaging cells are those that have been designed to limit homologous recombination that could lead to wildtype adenoviral particles. Cells that may be used to produce the adenoviral particles of the invention include the human embryonic kidney cell line 293 (Graham et al., J Gen. Virol. 36:59-72 (1977)), the human embryonic retinoblast cell line PER.C6 (U.S. Pat. Nos. 5,994,128 and 6,033,908; Fallaux et al., Hum. Gene Ther. 9: 1909-1917 (1998)), and the human cervical tumor-derived cell line HeLa-S3 (U.S. Pat Applic 60/463,143).

The present invention contemplates the use of all adenoviral serotypes to construct the adenoviral vectors and virus particles according to the present invention. In one embodiment, the adenoviral nucleic acid backbone is derived from adenovirus serotype 2(Ad2), 5 (Ad5) or 35 (Ad35), although other serotype adenoviral vectors can be employed. Adenoviral stocks that can be employed according to the invention include any adenovirus serotype. A large number of Adenovirus serotypes are currently available from American Type Culture Collection (ATCC, Manassas, Va.), and the invention includes any serotype of adenovirus available from any source including those serotypes listed in Table 1. The adenoviruses that can be employed according to the invention may be of human or non-human origin. For instance, an adenovirus can be of subgroup A (e.g., serotypes 12, 18, 31), subgroup B (e.g., serotypes 3, 7, 11, 14, 16, 21, 34, 35), subgroup C (e.g., serotypes 1, 2, 5, 6), subgroup D (e.g., serotypes 8, 9, 10, 13, 15, 17, 19, 20, 22-30, 32, 33, 36-39, 4247), subgroup E (serotype 4), subgroup F (serotype 40, 41), or any other adenoviral serotype. Throughout the specification reference is made to specific nucleotides in adenovirus type 5. One skilled in the art can determine the corresponding nucleotides in other serotypes and therefore construct similar adenoviral vectors in other adenovirus serotypes.

TABLE 1
Examples Of Human And Animal Adenoviruses Including The
American Type Culture Collection Catalog # For A
Representative Virus Of The Respective Classification

| Adenovirus Type | ATCC # |
|---|---|
| Adenovirus Type 21 | ATCC VR-1099 |
| SA18 (Simian adenovirus 18) | ATCC VR-943 |
| SA17 (Simian adenovirus 17) | ATCC VR-942 |
| Adenovirus Type 47 | ATCC VR-1309 |
| Adenovirus Type 44 | ATCC VR-1306 |
| Avian adenovirus Type 4 | ATCC VR-829 |
| Avian adenovirus Type 5 | ATCC VR-830 |
| Avian adenovirus Type 7 | ATCC VR-832 |
| Avian adenovirus Type 8 | ATCC VR-833 |
| Avian adenovirus Type 9 | ATCC VR-834 |
| Avian adenovirus Type 10 | ATCC VR-835 |
| Avian adenovirus Type 2 | ATCC VR-827 |
| Adenovirus Type 45 | ATCC VR-1307 |
| Adenovirus Type 38 | ATCC VR-988 |
| Adenovirus Type 46 | ATCC VR-1308 |
| Simian adenovirus | ATCC VR-541 |
| SA7 (Simian adenovirus 16) | ATCC VR-941 |
| Frog adenovirus (FAV-1) | ATCC VR-896 |
| Adenovirus type 48 (candidate) | ATCC VR-1406 |
| Adenovirus Type 42 | ATCC VR-1304 |
| Adenovirus Type 49 (candidate) | ATCC VR-1407 |
| Adenovirus Type 43 | ATCC VR-1305 |
| Avian adenovirus Type 6 | ATCC VR-831 |
| Avian adenovirus Type 3 | |
| Bovine adenovirus Type 3 | ATCC VR-639 |
| Bovine adenovirus Type 6 | ATCC VR-642 |
| Canine adenovirus | ATCC VR-800 |
| Bovine adenovirus Type 5 | ATCC VR-641 |
| Adenovirus Type 36 | ATCC VR-913 |
| Ovine adenovirus type 5 | ATCC VR-1343 |
| Adenovirus Type 29 | ATCC VR-272 |
| Swine adenovirus | ATCC VR-359 |
| Bovine adenovirus Type 4 | ATCC VR-640 |
| Bovine adenovirus Type 8 | ATCC VR-769 |
| Bovine adenovirus Type 7 | ATCC VR-768 |
| Adeno-associated virus Type2 (AAV-2H) | ATCC VR-680 |
| Adenovirus Type 4 | ATCC VR-4 |
| Adeno-associated virus Type3 (AAV-3H) | ATCC VR-681 |
| Peromyscus adenovirus | ATCC VR-528 |
| Adenovirus Type 15 | ATCC VR-661 |
| Adenovirus Type 20 | ATCC VR-662 |
| Chimpanzee adenovirus | ATCC VR-593 |
| Adenovirus Type 31 | ATCC VR-357 |
| Adenovirus Type 25 | ATCC VR-223 |
| Chimpanzee adenovirus | ATCC VR-592 |
| Chimpanzee adenovirus | ATCC VR-591 |
| Adenovirus Type 26 | ATCC VR-224 |
| Adenovirus Type 19 | ATCC VR-254 |
| Adenovirus Type 23 | ATCC VR-258 |
| Adenovirus Type 28 | ATCC VR-226 |
| Adenovirus Type 6 | ATCC VR-6 |
| Adenovirus Type 2 Antiserum: | ATCC VR-1079 |
| Adenovirus Type 6 | ATCC VR-1083 |
| Ovine adenovirus Type 6 | ATCC VR-1340 |
| Adenovirus Type 3 | ATCC VR-847 |
| Adenovirus Type 7 | ATCC VR-7 |
| Adenovirus Type 39 | ATCC VR-932 |
| Adenovirus Type 3 | ATCC VR-3 |
| Bovine adenovirus Type 1 | ATCC VR-313 |
| Adenovirus Type 14 | ATCC VR-15 |
| Adenovirus Type 1 | ATCC VR-1078 |
| Adenovirus Type 21 | ATCC VR-256 |
| Adenovirus Type 18 | ATCC VR-1095 |
| Baboon adenovirus | ATCC VR-275 |
| Adenovirus Type 10 | ATCC VR-11 |
| Adenovirus Type 33 | ATCC VR-626 |
| Adenovirus Type 34 | ATCC VR-716 |
| Adenovirus Type 15 | ATCC VR-16 |
| Adenovirus Type 22 | ATCC VR-257 |
| Adenovirus Type 24 | ATCC VR-259 |
| Adenovirus Type 17 | ATCC VR-1094 |
| Adenovirus Type 4 | ATCC VR-1081 |
| Adenovirus Type 16 | ATCC VR-17 |
| Adenovirus Type 17 | ATCC VR-18 |
| Adenovirus Type 16 | ATCC VR-1093 |
| Bovine adenovirus Type 2 | ATCC VR-314 |
| SV-30 | ATCC VR-203 |
| Adenovirus Type 32 | ATCC VR-625 |
| Adenovirus Type 20 | ATCC VR-255 |
| Adenovirus Type 13 | ATCC VR-14 |
| Adenovirus Type 14 | ATCC VR-1091 |
| Adenovirus Type 18 | ATCC VR-19 |
| SV-39 | ATCC VR-353 |
| Adenovirus Type 11 | ATCC VR-849 |
| Duck adenovirus (Egg drop syndrome) | ATCC VR-921 |
| Adenovirus Type 1 | ATCC VR-1 |
| Chimpanzee adenovirus | ATCC VR-594 |
| Adenovirus Type 15 | ATCC VR-1092 |
| Adenovirus Type 13 | ATCC VR-1090 |
| Adenovirus Type 8 | ATCC VR-1368 |
| SV-31 | ATCC VR-204 |
| Adenovirus Type 9 | ATCC VR-1086 |
| Mouse adenovirus | ATCC VR-550 |
| Adenovirus Type 9 | ATCC VR-10 |
| Adenovirus Type 41 | ATCC VR-930 |
| C1 | ATCC VR-20 |
| Adenovirus Type 40 | ATCC VR-931 |
| Adenovirus Type 37 | ATCC VR-929 |
| Marble spleen disease virus | |
| Adenovirus Type 35 | ATCC VR-718 |
| SV-32 (M3) | ATCC VR-205 |
| Adenovirus Type 28 | ATCC VR-1106 |
| Adenovirus Type 10 | ATCC VR-1087 |
| Adenovirus Type 20 | ATCC VR-1097 |
| Adenovirus Type 21 | ATCC VR-1098 |
| Adenovirus Type 25 | ATCC VR-1103 |
| Adenovirus Type 26 | ATCC VR-1104 |
| Adenovirus Type 31 | ATCC VR-1109 |
| Adenovirus Type 19 | ATCC VR-1096 |
| SV-36 | ATCC VR-208 |
| SV-38 | ATCC VR-355 |
| SV-25 (M8) | ATCC VR-201 |
| SV-15 (M4) | ATCC VR-197 |
| Adenovirus Type 22 | ATCC VR-1100 |
| SV-23 (M2) | ATCC VR-200 |
| Adenovirus Type 11 | ATCC VR-12 |
| Adenovirus Type 24 | ATCC VR-1102 |
| Avian adenovirus Type 1 | |
| SV-11 (M5) | ATCC VR-196 |
| Adenovirus Type 5 | ATCC VR-5 |
| Adenovirus Type 23 | ATCC VR-1101 |
| SV-27 (M9) | ATCC VR-202 |
| Avian adenovirus Type 2 (GAL) | ATCC VR-280 |
| SV-1 (M1) | ATCC VR-195 |
| SV-17 (M6) | ATCC VR-198 |
| Adenovirus Type 29 | ATCC VR-1107 |
| Adenovirus Type 2 | ATCC VR-846 |
| SV-34 | ATCC VR-207 |
| SV-20 (M7) | ATCC VR-199 |
| SV-37 | ATCC VR-209 |
| SV-33 (M10) | ATCC VR-206 |
| Avian adeno-associated virus | ATCC VR-865 |
| Adeno-associated (satellite) virus Type 4 | ATCC VR-646 |
| Adenovirus Type 30 | ATCC VR-273 |
| Adeno-associated (satellite) virus Type1 | ATCC VR-645 |
| Infectious canine hepatitis (Rubarth's disease) | |
| Adenovirus Type 27 | ATCC VR-1105 |
| Adenovirus Type 12 | ATCC VR-863 |
| Adeno-associated virus Type 2 | |
| Adenovirus Type 7a | ATCC VR-848 |

In one aspect the present invention provides a recombinant adenoviral vector comprising an adenoviral nucleic acid backbone, wherein said nucleic acid backbone comprises in sequential order: a left ITR, a termination signal sequence, a cancer specific FEN1 TRE of the invention that is operatively linked to a first gene essential for replication of the recombinant adenoviral vector, and a right ITR.

In another aspect the present invention provides a recombinant adenoviral vector comprising an adenoviral nucleic acid backbone, wherein said nucleic acid backbone comprises in sequential order: a left ITR, a termination signal sequence, a FEN1 TRE of the invention that is operatively linked to a first gene essential for replication of the recombinant adenoviral vector, an adenoviral packaging signal, and a right ITR.

In another aspect, the present invention provides a recombinant adenoviral vector comprising an adenoviral nucleic acid backbone, wherein said nucleic acid backbone comprises in sequential order: a left ITR, an adenoviral packaging signal, a first TRE operatively linked to a first gene essential for replication of the recombinant adenoviral vector, a TRE operatively linked to a second gene essential for replication (wherein the first and second cancer specific regulatory regions are not the same), and a right ITR.

In yet another aspect, the present invention provides a recombinant adenoviral vector comprising an adenoviral nucleic acid backbone, wherein said nucleic acid backbone comprises in sequential order: a left ITR, an adenoviral packaging signal, a TRE operatively linked to a first gene essential for replication of the recombinant adenoviral vector, a second TRE operatively linked to a transgene and a right ITR.

The first and second TREs may be cancer specific regulatory regions and may or may not be essentially the same. The vector may or may not have a termination signal sequence 5′ to the first cancer specific regulatory region and may or may not have a relocated packaging signal. In one embodiment, the first cancer specific regulatory region is a FEN1 TRE operatively linked to E1a and the regulatory region is an hTERT TRE or an E2F-1 TRE operatively linked to E1b or E4. In another embodiment, the first cancer specific regulatory region is an hTERT TRE or an E2F-1 TRE operatively linked to E1a and the second cancer specific regulatory region is a FEN1 TRE operatively linked to E1b or E4.

The recombinant adenoviral vectors of this invention are useful as therapeutics for treatment of cancer. As demonstrated herein, FEN1 is overexpressed in tumor cells. The vectors of the invention exhibit a favorable toxicity profile, which is clinically acceptable for the condition to be treated. Without wishing to be limited by theoretical considerations, the specific regulation of viral replication by a FEN1 TRE, which optionally may be shielded from read-through transcription by an upstream termination signal sequence, avoids toxicity that would occur if it replicated in non-target tissues, allowing for the favorable efficacy/toxicity profile.

In one embodiment, the recombinant viral vector of the invention comprises a termination signal sequence. A termination signal sequence may also be placed before (5′) any TRE in the vector. In one embodiment, the terminal signal sequence is placed before a heterologous TRE operatively linked to the a E1b or E4 gene.

In another embodiment, the recombinant viral vector further comprises a deletion upstream of the termination signal sequence, such as a deletion between nucleotides 103 and 551 of the adenoviral type 5 backbone or corresponding positions in other serotypes. A deletion in the packaging signal 5′ to the termination signal sequence may be such that the packaging signal becomes non-functional. In one embodiment, the deletion comprises a deletion 5′ to the termination signal sequence wherein the deletion spans at least the nucleotides 189 to 551. In another embodiment the deletion comprises a deletion 5′ to the termination signal sequence wherein the deletion spans at least nucleotides 103 to 551 (FIG. 2 of WO 02/067861 and WO 02/068627). In a further embodiment, the packaging signal is located (i.e. re-inserted) downstream of the FEN1 TRE-linked gene essential for replication.

Transgenes

To further enhance therapeutic efficacy, the vectors of the invention may include one or more transgenes that have a therapeutic effect, such as enhancing cytotoxicity so as to eliminate unwanted target cells. The transgene may be under the transcriptional control of a cancer-specific TRE, e.g. a FEN1 TRE. The transgene may be regulated independently of the adenovirus gene regulation, e.g. having separate promoters, which may be the same or different, or may be coordinately regulated, e.g. having a single promoter in conjunction with an IRES or a self-processing cleavage sequence, such as a 2A sequence. In this approach expression of the E1A and E1B genes may be linked by an IRES between the E1A and E1B genes. In the construction of this virus, the endogenous E1B promoter elements are removed and replaced with the IRES element. Therefore both E1A and E1B expression are under the control of the inducer responsive promoter element. As an IRES alternative, the 2A peptide sequence derived foot and mouth disease virus (FMDV) could be used in place of the IRES sequence (as described in Furler S et al., Gene Ther. 2001 June;8(11):864-73) to provide efficient bicistronic expression of both E1A and a transgene.

In this way, various genetic capabilities may be introduced into target cells, particularly cancer cells. Alternatively, the vector may comprise a heterologous transgene encoding a therapeutic gene product under the control of a constitutive or inducible promoter. Numerous examples of constitutive and inducible promoters are known in the art and routinely employed in transgene expression in the context of viral or non-viral vectors. In this way, various genetic capabilities may be introduced into target cells. For example, in certain instances, it may be desirable to enhance the degree therapeutic efficacy by enhancing the rate of cytotoxic activity. This could be accomplished by coupling the cancer cell-specific TRE activity with expression of, one or more metabolic enzymes such as HSV-tk, nitroreductase, cytochrome P450 or cytosine deaminase (CD) which render cells capable of metabolizing 5-fluorocytosine (5-FC) to the chemotherapeutic agent 5-fluorouracil (5-FU), carboxylesterase (CA), deoxycytidine kinase (dCK), purine nucleoside phosphorylase (PNP), thymidine phosphorylase (TP), thymidine kinase (TK) or xanthine-guanine phosphoribosyl transferase (XGPRT). This type of transgene may also be used to confer a bystander effect.

Any gene or coding sequence of therapeutic relevance can be used in the practice of the invention. For example, genes encoding immunogenic polypeptides, toxins, immunotoxins and cytokines are useful in the practice of the invention. Additional transgenes that may be introduced into a vector of the invention include a factor capable of initiating apoptosis, antisense or ribozymes, which among other capabilities may be directed to mRNAs encoding proteins essential for proliferation, such as structural proteins, transcription factors, polymerases, etc., viral or other pathogenic proteins, where the pathogen proliferates intracellularly, cytotoxic proteins, e.g., the chains of diphtheria, ricin, abrin, etc., genes that encode an engineered cytoplasmic variant of a nuclease (e.g., RNase A) or protease (e.g., trypsin, papain, proteinase K, carboxypeptidase, etc.), chemokines, such as MCP3 alpha or MIP-1, pore-forming proteins derived from viruses, bacteria, or mammalian cells, fusogenic genes, chemotherapy sensitizing genes and radiation sensitizing genes.

Other genes of interest include cytokines, antigens, transmembrane proteins, and the like, such as IL-1, IL-2, IL-4, IL-5, IL-6, IL-10, IL-12, IL-18 or flt3, GM-CSF, G-CSF, M-CSF, IFN-α, -β, -γ, TNF-α, -β, TGF-α, -β, NGF, MDA-7 (Melanoma differentiation associated gene-7, mda-7/interleukin-24), and the like. Further examples include, proapoptotic genes such as Fas, Bax, Caspase, TRAIL, Fas ligands, nitric oxide synthase (NOS) and the like; fusion genes which can lead to cell fusion or facilitate cell fusion such as V22, VSV and the like; tumor suppressor gene such as p53, RB, p16, p17, W9 and the like; genes associated with the cell cycle and genes which encode anti-angiogenic proteins such as endostatin, angiostatin and the like.

Other opportunities for specific genetic modification include T cells, such as tumor infiltrating lymphocytes (TILs), where the TILs may be modified to enhance expansion, enhance cytotoxicity, reduce response to proliferation inhibitors, enhance expression of lymphokines, etc. One may also wish to enhance target cell vulnerability by providing for expression of specific surface membrane proteins, e.g., B7, SV40 T antigen mutants, etc.

Additional genes include the following: proteins that stimulate interactions with immune cells such as B7, CD28, MHC class I, MHC class II, TAPs, tumor-associated antigens such as immunogenic sequences from MART-1, gp 100(pmel-17), tyrosinase, tyrosinase-related protein 1, tyrosinase-related protein 2, melanocyte-stimulating hormone receptor, MAGEI, MAGE2, MAGE3, MAGE12, BAGE, GAGE, NY-ESO-1, β-catenin, MUM-1, CDK-4, caspase 8, KIA 0205, HLA-A2R1701, α-fetoprotein, telomerase catalytic protein, G-250, MUC-1, carcinoembryonic protein, p53, Her2/neu, triosephosphate isomerase, CDC-27, LDLR-FUT, telomerase reverse transcriptase, PSMA, cDNAs of antibodies that block inhibitory signals (CTLA4 blockade), chemokines (MIPIα, MIP3α, CCR7 ligand, and calreticulin), anti-angiogenic genes include, but are not limited to, genes that encode METH-1, METH -2, TrpRS fragments, proliferin-related protein, prolactin fragment, PEDF, vasostatin, various fragments of extracellular matrix proteins and growth factor/cytokine inhibitors, various fragments of extracellular matrix proteins which include, but are not limited to, angiostatin, endostatin, kininostatin, fibrinogen-E fragment, thrombospondin, tumstatin, canstatin, restin, growth factor/cytokine inhibitors which include, but are not limited to, VEGF/VEGFR antagonist, sFlt-I, sFlk, sNRPI, angiopoietin/tie antagonist, sTie-2, chemokines (IP-10, PF4, Gro-beta, IFN-gamma (Mig), IFNα, FGF/FGFR antagonist (sFGFR), Ephrin/Eph antagonist (sEphB4 and sephrinB2), PDGF, TGFβ and IGF-I. Genes suitable for use in the practice of the invention can encode enzymes (such as, for example, urease, renin, thrombin, metalloproteases, nitric oxide synthase, superoxide dismutase, catalase and others known to those of skill in the art), enzyme inhibitors (such as, for example, alpha1-antitrypsin, antithrombin III, cellular or viral protease inhibitors, plasminogen activator inhibitor-1, tissue inhibitor of metalloproteases, etc.), the cystic fibrosis transmembrane conductance regulator (CFTR) protein, insulin, dystrophin, or a Major Histocompatibility Complex (MHC) antigen of class I or II. Also useful are genes encoding polypeptides that can modulate/regulate expression of corresponding genes, polypeptides capable of inhibiting a bacterial, parasitic or viral infection or its development (for example, antigenic polypeptides, antigenic epitopes, and transdominant protein variants inhibiting the action of a native protein by competition), apoptosis inducers or inhibitors (for example, Bax, Bc12, Bc1X and others known to those of skill in the art), cytostatic agents (e.g., p21, p16, Rb, etc.), apolipoproteins (e.g., ApoAI, ApoAIV, ApoE, etc.), oxygen radical scavengers, polypeptides having an anti-tumor effect, antibodies, toxins, immunotoxins, markers (e.g., beta-galactosidase, luciferase, etc.) or any other genes of interest that are recognized in the art as being useful for treatment or prevention of a clinical condition. Further therapeutic genes include a polypeptide which inhibits cellular division or signal transduction, a tumor suppressor gene (such as, for example, p53, Rb, p73), a polypeptide which activates the host immune system, a tumor-associated antigen (e.g., MUC-1, BRCA-1, an HPV early or late antigen such as E6, E7, L1, L2, etc), optionally in combination with a cytokine gene.

The invention further comprises combinations of two or more transgenes with synergistic, complementary and/or nonoverlapping toxicities and methods of action. The resulting adenovirus would retain the viral oncolytic functions and would, for example, additionally have the ability to induce immune and anti-angiogenic responses, etc.

In the vectors of the invention, a transgene/therapeutic gene or coding sequence therefor is under the control of a FEN1 or other suitable promoter. Suitable promoters that may be employed include, but are not limited to, adenoviral promoters, such as the adenoviral major late promoter and/or the E3 promoter; promoters such as the cytomegalovirus (CMV) promoter; the Rous Sarcoma Virus (RSV) promoter; inducible promoters, such as the MMT promoter, the metallothionein promoter; heat shock promoters; the albumin promoter; the ApoAI promoter; and a tissue-specific TRE such as those disclosed in WO 99/25860.

Therapeutic Methods

An effective amount of a FEN-1 TRE-containing vector is administered to a patient as a composition in a pharmaceutically acceptable excipient, including, but not limited to, saline solutions, suitable buffers, preservatives, stabilizers, and may be administered in conjunction with suitable agents such as antiemetics. An effective amount is an amount sufficient to effect beneficial or desired results, including clinical results. An effective amount can be administered in one or more administrations. For purposes of this invention, an effective amount of vector is an amount that is sufficient to palliate, ameliorate, stabilize, reverse, slow or delay the progression of the disease state. Some individuals are refractory to these treatments, and it is understood that the methods encompass administration to these individuals. The amount to be given will be determined by the condition of the individual, the extent of disease, the route of administration, how many doses will be administered, and the desired objective.

Delivery of vectors of the invention is generally accomplished by either site-specific injection or intravenous injection. Site-specific injections of vector may include, for example, injections into tumors, as well as intraperitoneal, intrapleural, intrathecal, intra-arterial, subcutaneous or topical application. These methods are easily accommodated in treatments using the combination of vectors and chemotherapeutic agents.

Viral vectors may be delivered to the target cell in a variety of ways, including, but not limited to, liposomes, general transfection methods that are well known in the art (such as calcium phosphate precipitation or electroporation), direct injection, and intravenous infusion. The means of delivery will depend in large part on the particular vector (including its form) as well as the type and location of the target cells (i.e., whether the cells are in vitro or in vivo).

If used as a packaged adenovirus, adenovirus vectors may be administered in an appropriate physiologically acceptable carrier at a dose of about 10⁴ to about 10¹⁴ viral particles. If administered as a polynucleotide construct (i.e., not packaged as a virus) about 0.01 ug to about 1000 ug of an adenoviral vector can be administered. The exact dosage to be administered is dependent upon a variety of factors including the age, weight, and sex of the patient, and the size and severity of the tumor being treated. The adenoviral vector(s) may be administered one or more times, depending upon the intended use and the immune response potential of the host, and may also be administered as multiple, simultaneous injections. If an immune response is undesirable, the immune response may be diminished by employing a variety of immunosuppressants, or by employing a technique such as an immunoadsorption procedure (e.g., immunoapheresis) that removes adenovirus antibody from the blood, so as to permit repetitive administration, without a strong immune response. If packaged as another viral form, such as HSV, an amount to be administered is based on standard knowledge about that particular virus (which is readily obtainable from, for example, published literature) and can be determined empirically.

In one embodiment the host organism is a human patient. For human patients, if a therapeutic gene is included in the vector, the therapeutic gene may be of human origin although genes of closely related species that exhibit high homology and biologically identical or equivalent function in humans may be used if the gene does not produce an adverse immune reaction in the recipient. A therapeutic active amount of a nucleic acid sequence or a therapeutic gene is an amount effective at dosages and for a period of time necessary to achieve the desired result. This amount may vary according to various factors including but not limited to sex, age, weight of a subject, and the like.

Embodiments of the present invention include methods for the administration of combinations of a cancer-specific vector and a second anti-neoplastic therapy, which may include radiation, administration of an anti-neoplastic agent, etc., to an individual with neoplasia, as detailed in U.S. Application 20030068307. The cancer-specific vector and anti-neoplastic (chemotherapeutic) agent may be administered simultaneously or sequentially, with various time intervals for sequential administration. In some embodiments, an effective amount of vector and an effective amount of at least one chemotherapeutic agent are combined with a suitable excipient and/or buffer solutions and administered simultaneously from the same solution by any of the methods listed herein or those known in the art. This may be applicable when the chemotherapeutic agent does not compromise the viability and/or activity of the vector itself.

Where more than one chemotherapeutic agent is administered, the agents may be administered together in the same composition; sequentially in any order; or, alternatively, administered simultaneously in different compositions. If the agents are administered sequentially, administration may further comprise a time delay. Sequential administration may be in any order, and accordingly encompasses the administration of an effective amount of an vector first, followed by the administration of an effective amount of the chemotherapeutic agent. The interval between administration of the cancer-specific vector and chemotherapeutic agent may be in terms of at least (or, alternatively, less than) minutes, hours, or days. Sequential administration also encompasses administration of a chosen chemotherapeutic agent followed by the administration of the vector. The interval between administration may be in terms of at least (or, alternatively, less than) minutes, hours, or days.

Administration of the above-described methods may also include repeat doses or courses of a cancer-specific vector and chemotherapeutic agent depending, inter alia, upon the individual's response and the characteristics of the individual's disease. Repeat doses may be undertaken immediately following the first course of treatment (i.e., within one day), or after an interval of days, weeks or months to achieve and/or maintain suppression of tumor growth. A particular course of treatment according to the above-described methods, for example, combined cancer-specific vector and chemotherapy, may later be followed by a course of combined radiation and cancer-specific vector therapy.

Anti-neoplastic (chemotherapeutic) agents include those from each of the major classes of chemotherapeutics, including but not limited to: alkylating agents, alkaloids, antimetabolites, anti-tumor antibiotics, nitrosoureas, hormonal agonists/antagonists and analogs, immunomodulators, photosensitizers, enzymes and others. In some embodiments, the antineoplastic is an alkaloid, an antimetabolite, an antibiotic or an alkylating agent. In certain embodiments the antineoplastic agents include, for example, thiotepa, interferon alpha-2a, and the M-VAC combination (methotrexate-vinblastine, doxorubicin, cyclophosphamide). Preferred antineoplastic agents include, for example, 5-fluorouracil, cisplatin, 5-azacytidine, and gemcitabine. Particularly preferred embodiments include, but are not limited to, 5-fluorouracil, gemcitabine, doxorubicin, miroxantrone, mitomycin, dacarbazine, carmustine, vinblastine, lomustine, tamoxifen, docetaxel, paclitaxel or cisplatin. The specific choice of both the chemotherapeutic agent(s) is dependent upon, inter alia, the characteristics of the disease to be treated. These characteristics include, but are not limited to, location of the tumor, stage of the disease and the individual's response to previous treatments, if any.

In addition to the use of single chemotherapeutic agent in combination with a particular cancer-specific vector, the invention also includes the use of more than one agent in conjunction with the cancer-specific vector. These combinations of antineoplastics when used to treat neoplasia are often referred to as combination chemotherapy and are often part of a combined modality treatment which may also include surgery and/or radiation, depending on the characteristics of an individual's cancer. It is contemplated that the cancer-specific vector/chemotherapy of the present invention can also be used as part of a combined modality treatment program.

There are a variety of delivery methods for the administration of antineoplastic agents, which are well known in the art, including oral and parenteral methods. There are a number of drawbacks to oral administration for a large number of antineoplastic agents, including low bioavailability, irritation of the digestive tract and the necessity of remembering to administer complicated combinations of drugs. The majority of parenteral administration of chemotherapeutic agents is intravenously, as intramuscular and subcutaneous injection often leads to irritation or damage to the tissue. Regional variations of parenteral injections include intra-arterial, intravesical, intra-tumor, intrathecal, intrapleural, intraperitoneal and intracavity injections.

Delivery methods for chemotherapeutic agents include intravenous, intraparenteral and intraperitoneal methods as well as oral administration. Intravenous methods also include delivery through a vein of the extremities as well as including more site specific delivery, such as an intravenous drip into the portal vein. Other intraparenteral methods of delivery include direct injections of an antineoplastic solution, for example, subcutaneously, intracavity or intra-tumor.

Assessment of the efficacy of a particular treatment regimen may be determined by any of the techniques known in the art, including diagnostic methods such as imaging techniques, analysis of serum tumor markers, biopsy, the presence, absence or amelioration of tumor associated symptoms. It will be understood that a given treatment regime may be modified, as appropriate, to maximize efficacy.

In a further aspect of the invention, a pharmaceutical composition comprising the recombinant viral vectors and/or viral particles of the invention and a pharmaceutically acceptable carrier is provided. Such compositions, which can comprise an effective amount of cancer-specific vector and/or viral particles of the invention in a pharmaceutically acceptable carrier, are suitable for local or systemic administration to individuals, in unit dosage forms, sterile parenteral solutions or suspensions, sterile non-parenteral solutions or oral solutions or suspensions, oil in water or water in oil emulsions and the like. Formulations for parenteral and non-parenteral drug delivery are known in the art. Compositions also include lyophilized and/or reconstituted forms of the cancer-specific vector or particles of the invention. Acceptable pharmaceutical carriers are, for example, saline solution, protamine sulfate (Elkins-Sinn, Inc., Cherry Hill, N.J.), water, aqueous buffers, such as phosphate buffers and Tris buffers, or Polybrene (Sigma Chemical, St. Louis Mo.) and phosphate-buffered saline and sucrose. The selection of a suitable pharmaceutical carrier is deemed to be apparent to those skilled in the art from the teachings contained herein. These solutions are sterile and generally free of particulate matter other than the desired cancer-specific vector. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. Excipients that enhance uptake of the cancer-specific vector by cells may be included.

Screening Agents And Assays

The invention also provides for screening candidate drugs to identify agents useful for modulating the expression of FEN1 in cancer tissue and useful for treating cancer. Appropriate host cells are those in which the regulatory region of FEN1 is capable of functioning. In one embodiment, a FEN1 TRE is used to make a variety of expression vectors to express a marker that can then be used in screening assays. The expression vectors may be either self-replicating extrachromosomal vectors or vectors that integrate into a host genome. Generally, these expression vectors include a transcriptional and translational regulatory nucleic acid sequence of FEN1 operatively linked to a nucleic acid encoding a marker. The marker may be any protein that can be readily detected. It may be detected on the basis of light emission, such as luciferase and GFP, color, such as β-galactosidase, enzyme activity, such as alkaline phosphatase or antibody reaction, such as a protein for which an antibody exists. In addition, the marker system may be a vector or viral particle of the present invention.

The present invention also provides methods for screening compounds that are useful for modulating the expression of FEN1 in cancer tissue. In one embodiment, the method of screening compounds includes comparing the level of FEN1 expression in the absence of the compound to the level of expression in the presence of the drug candidate, wherein the concentration of the compound can vary when present, and wherein the comparison can occur after addition or removal of the compound. The method may utilize eukaryotic or prokaryotic host cells that are stably transformed with recombinant polynucleotides comprising a regulatory region of the FEN1 gene operatively linked to a nucleic acid sequence encoding a product that can be detected. A candidate compound is added to the host cells and the expression of the detectable product is compared to a control.

The present invention further provides a method that utilizes host cells transduced with a viral vector comprising a FEN1 TRE of the invention operatively linked to an essential viral gene, e.g., for screening compounds useful for modulating the expression of FEN1 in cancer tissue. According to this method, a candidate compound is added to the host cells and expression of the essential viral gene or viral replication is detected and compared to a control.

The various methods of the invention will be described below. Although particular methods of tumor suppression are exemplified in the discussion below, it is understood that any of a number of alternative methods, including those described above are equally applicable and suitable for use in practicing the invention. It will also be understood that an evaluation of the vectors and methods of the invention may be carried out using procedures standard in the art, including the diagnostic and assessment methods described above.

In one embodiment, the viral vector or particle is used to assess the modulation of the FEN1 TRE. According to this embodiment, an effective amount of the viral vectors or viral particles of the invention is contacted with said cell population under conditions where the viral vectors or particles can transduce the neoplastic cells in the cell population, replicate, and kill the neoplastic cells. The candidate agent is either present in the culture medium for the test sample or absent for the control sample. The LD50 of the viral vectors or particles in the presence and absence of the candidate agent is compared to identify the candidate agents that modulate the expression of the FEN1 gene. If the level of expression is different as compared to similar viral vector controls lacking the FEN1 TRE, the candidate agent is capable of modulating the expression of FEN1 and is a candidate for treating cancers involving this gene and for further development of active agents on the basis of the candidate agent so identified.

In a second embodiment, the candidate agent is added to host cells containing the expression vector and the level of expression of the marker is compared with a control. If the level of expression is different, the candidate agent is capable of modulating the expression of FEN1 and is a candidate for treating cancers involving this gene and for further development of active agents on the basis of the candidate agent so identified.

Active agents so identified may also be used in combination treatments with a cancer-specific vector of the invention.

Having identified the FEN1 gene as being associated with cancer, a variety of assays may be executed. In an embodiment, assays may be run on an individual gene or protein level. That is, having identified a gene as up-regulated in cancer, candidate bioactive agents may be screened to modulate this gene's response; preferably to down-regulate the gene, although in some circumstances to up regulate the gene. “Modulation” thus includes both an increase and a decrease in gene expression. The preferred amount of modulation will depend on the original change of the gene expression in normal versus tumor tissue, with changes of at least 10%, preferably 50%, more preferably 100-300%, and in some embodiments 300-1000% or greater. Thus, if a gene exhibits a 4 fold increase in tumor compared to normal tissue, a decrease of about four fold is desired; a 10 fold decrease in tumor compared to normal tissue gives a 10 fold increase in expression for a candidate agent is desired.

Candidate agents encompass numerous chemical classes, though typically they are organic molecules, preferably small organic compounds having a molecular weight of more than 100 and less than about 2,500 daltons. Preferred small molecules are less than 2000, or less than 1500 or less than 1000 or less than 500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally; natural or synthetically produced libraries and compounds are readily modified through conventional chemical, physical and biochemical means. Known pharmacological agents may be subjected to directed or random chemical modifications, such as acylation, alkylation, esterification, and amidification to produce structural analogs.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. The following examples are offered by way of illustration and not by way of limitation.

### EXPERIMENTAL

### Example 1

### Identification Of Cancer-Selective Genes By Gene Expression Profiling With Tumor/Normal Tissue Microarray Databases

A tumor/normal tissue microarray database including prostate tumors, normal prostates, colon tumors, normal colons, as well as normal lungs, normal livers, normal kidneys and one normal heart were analyzed. RNA samples were hybridized with more than 200,000 different oligonucleotide probes on Affymetrix human U95A v.2 chips. The transcript levels of more than 8000 known genes in the human genome were profiled for each sample. Differential expression of the genes (expressed as “average difference values”) in tumor and normal tissues was determined using an algorithm that ranks the genes by criteria designed to identify genes that show low to high expression levels in a majority of samples from a cancer of interest, and that show an absence of expression in a majority of samples from non-target tissues. Non-target tissues include matched normal tissue for the particular cancer type (except prostate cancer), liver, lung, kidney and heart. These criteria were applied to the microarray data in Excel by setting the following parameters by which to rank the candidate gene average difference values: a mean level of expression in the tumor samples greater than 200 relative units, a mean level of expression in the normal matched tissue of less than 40 (except for prostate cancer), a mean level of expression in non-target tissues of less than 40, and having the above criteria met in >50% of the tumor and normal tissue samples.

The candidates were further evaluated for their expression levels in different cell lines to select cell lines that can be used as positive and negative controls for expression. The genes that fulfilled the criteria for differential expression both in tumor tissues as well as in particular cell lines were determined to be candidate genes.

In an exemplary study carried out according to the above, a tumor/normal tissue microarray database including 24 prostate tumors, 9 normal prostates, 21 colon tumors, 5 normal colons, as well as 5 normal lungs, 5 normal livers, 4 normal kidneys and one normal heart was analyzed. The majority of the genes were expressed in at least one cell line representative of the original tumor, but not expressed in at least one other cell line. Characteristics of a cancer-associated gene identified from the microarray data mining, FEN1, are shown in Table 2 below.

TABLE 2
Cancer Selective FEN1 Gene Identified By Expression Profiling

| | | | | | |
|---|---|---|---|---|---|
| Gene | | | | | |
| Symbol | | | | | |
| probe | | | GeneBank | Unigen | |
| set ID | U95a chip | Attributes | Acc.# | ID | mRNA |

| | | | | |
|---|---|---|---|---|
| FEN1 | 1516_g_at | Flap- | X76771 | NM_004111 |
| | | endonuclease | Hs.4756 | |
| | | 1 | | |

### Example 2

### Validation Of Selective Gene Expression In Tumor Target Versus Non-Target and Normal Cell Lines By Semi-Quantitative rt-PCR

Semi-quantitative rt-PCR is used to validate the differential expression of a candidate gene identified by micro array profiling. cDNA is prepared using RETROscript kit manufactured by Ambion Ltd (Austin, Tex.) from each cell line. Primers used to amplify the FEN1 cDNA are: sense, 5′-GCAAGAAGGCCACAGAGGTACT-3′ (SEQ ID NO:4) and antisense, 5′-GATTGCCAGGTGAACATCACCATC-3′; (SEQ ID NO:5). Multiplex PCR amplification was carried out in which the FEN1 cDNA was co-amplified with ribosomal 18s cDNA in the presence of FEN1-specific primers and 18s-specific primers provided in QuantumRNA 18s Internal Standard kit manufactured by Ambion Ltd (Austin, Tex.). The image intensity of the FEN1 transcript was normalized to the intensity of 18s transcript so that the level of FEN1 expression was semi-quantified and comparable among different cell lines. Sets of cell lines originally grouped into positive/negative cell lines by gene expression profiling were examined for their expression levels by the semi-quantitative rt-PCR method. The sensitivity by PCR amplification is higher than the one by microarray. This feature allows a high level of stringency in determining the negative cell lines. Thus, the PCR-identified differential pattern is used as a guideline for selecting positive/negative cell lines to be used as target/non-target cell lines to screen with the corresponding candidate oncolytic vectors for in vitro tumor-selective killing effects.

In an exemplary study carried out according to the above, the differential patterns detected by rt-PCR amplification were determined to be in reasonable agreement with the patterns identified by the expression profiling (see Table 3 below). In some cases, rt-PCR amplification detected a low level of expression that was not detected by the expression profiling. Nevertheless, the differential pattern maintained between the positive and negative cell lines. The FEN1 gene was expressed in multiple tumor cell lines. Two cell lines each from colon, lung and prostate cancer indication were selected as FEN1-positive cell lines: SW620, HT29, H446, H69, and C4-2. FEN1 was marginally expressed in primary HAEC and Wi38 cells, and not expressed in HRE cells.

TABLE 3
FEN1-Positive/Negative Cell Lines By rt-PCR vs. Microarray¹

| | | Expression by | Expression by |
|---|---|---|---|
| Cell line | Cell type | Microarray | rt-PCR |
| SW620 | colon tumor | + | + |
| HT29 | colon tumor | + | + |
| H446 | small cell lung cancer | + | NA |
| H69 | small cell lung cancer | + | NA |
| Skmel28 | melanoma | + | + |
| LNCap | prostate tumor | + | + |
| C4-2 | prostate tumor | + | NA |
| Wi38 | human primary fibroblast | + | ± |
| HAEC | human aorta epithelial cell | NA | ± |
| HRE | human aorta epithelial cell | NA | − |
| Prostate | prostate tissue | ± | ± |

¹Expression levels were scored as: + expressed, ± low expression, − not expressed.
NA = data not available. Measurement in prostate tissue was included to indicate the expression level in a non-target tissue.

### Example 3

### Promoter Annotation And Sequence Determination

Several web-based computational tools are applied to assist the annotation of a promoter in the human genome. An exon map of the gene in the GenBank database (available on the web at http://www.ncbi.nih.gov/cgi-bin) is used to determine the 5′ end of an mRNA sequence. The first base pair of the exon 1 sequence usually indicates a transcription start site (TSS). The basal promoter region is generally defined as being within 500 bp upstream of the TSS. To include certain transcription factor binding sites further upstream of the basal promoter sequence, a region containing 1.9 to 2 kb upstream and 100 to 250 bp downstream of the TSS is retrieved from the NCBI human genome database. FEN1 is located in the human genome at Chromosome 11, contig NT_{—}030106. The sequence of the retrieved promoter region is shown as SEQ ID NO: 1.

To predict the functionality of the retrieved promoter sequence, the sequence is analyzed for basal promoter elements and transcription factor binding sites using the computational tool linked to TRANSFAC, a transcription factor binding site database available on the web at http://www.genomatix.de/cgi-binieldorado/mail.pl. Examples of criteria that are used to determine which of the promoter fragments to test for tumor-selectivity are: 1) the sequence preferably contains either a TATA box or an Ebox/GC rich region in the proximal region, and 2) common transcription factor binding sites preferably occur as a cluster with each other forming a particular pattern. As an example of the prediction result, the promoter contains an Ebox/GC rich region within 600 bp upstream of TSS, while it does not contain a TATA box sequence. In addition, two common transcription factor binding sites, AP2F and EGRF, cluster within 250 bp upstream of TSS in this promoter. These characteristic components provide evidence for the sequence to be a basal promoter region.

### Example 4

### Construction Of Oncolytic Adenoviral And Luciferase Reporter Vectors With A FEN1 Promoter Sequence

An approximate 2.3 kb sequence 5′-upstream of the transcription start site (TSS) of the FEN gene was isolated from human genomic DNA (Clontech #6550-1) by PCR amplification. The 2.3 kb fragment was cloned into two types of Ad5-based oncolytic vector backbones. The specific primer sequences (underlined) for generating the FEN promoter fragment are: sense with an Nhel restriction site, 5′-CATGCTGCTAGCCATGCGGTTATCAAGGAGCC-3′ (SEQ ID NO:6) with an EcoRV restriction site, 5′-TTGGATATCGACGTTCAGCCGCCTTCCAA-3′ (SEQ ID NO:7).

The 2.2 kb FEN PCR fragment was cloned into PCR2.1 by TA cloning (Invitrogen) to generate pCR2.1fen. The pCR2.1fen plasmid was then digested with Nhel and EcoRV and the 2.3 kb fragment is ligated into Nhel and EcoRV sites between SV40 polyadenylation signal and the adenoviral E1a coding region of pDL6pA (Jakubczak et al. Cancer Res. 2003 April 1;63(7):14909) to generate the pDL6pAFen left end shuttle vector. The pDL6pAFen plasmid was then cut with Asel and Blpl, and the 6.9 kb fragment containing the FEN promoter and E1A coding region was incorporated into BstBI digested Ad plasmid pAr13pAE2fF (US Patent Publication 20030104625) through homologous recombination by the method described in He et al. (1998) to generate infectious pAr13pAFenF plasmid for production of the virus Ar13pAFenF.

pDL5pAxp is an adenoviral shuttle plasmid that comprises in sequential order an Ad5 Left ITR and packaging signal (nt 1 to bp 361 from Ad5), a SV40 poly A signal and nts 552 to 8099 of Ad5. The E1A promoter and cap site are deleted in pDL5pAxp corresponding to nts 362 to 575 of Ad5. In other words, pDL5pAxp is an Ad5 left end shuttle plasmid that has an SV40 pA and several restriction sites cloned in place of the E1A promoter and cap site. pAr21pAF is a plasmid comprising in sequential order an Ad5 left ITR and packaging signal (nt 1 to bp 361 from Ad5), an SV40 poly A, a multiple cloning site, and essentially the rest of adenovirus 5 genome starting at the E1 coding region including a complete E3 region and again the E1A promoter and cap site are deleted (corresponding to a deletion of nts 362 to 575 of Ad5).

To generate a FEN shuttle vector with the packaging signal at the left end of the Ad5 genome, 2.2 kb BamHI/EcoRV fragment containing the FEN promoter sequence was isolated from pDL6pAFen and ligated to the 11.2 kb BamHI/Pmel fragment digested from pDL5pAxp to generate pDL5pAFen shuttle vector. The pDL5pFen vector was then digested with Asel and Sphl, and the 7.4 kb fragment containing the FEN promoter and E1A coding region was incorporated into BstBI digested pAr21pAF through homologous recombination (He et al., 1998) to generate infectious pAr21pAFenF plasmid for production of Ar21-1035 viral particles.

Both Ar13pAFenF and Ar21-1035 are oncolytic adenoviral vectors in which the E1a promoter is replaced with a human FEN TRE. The FEN TRE in the Ar21-1035 vector is located downstream of the adenoviral packaging signal/left ITR enhancer sequences (ψ) at the left end of the Ad5 genome. In Ar13pAFenF vector, the packaging signal/enhancer sequences were relocated to the right end of the Ad5 genome to reduce the potential coregulation with the FEN promoter.

To evaluate promoter selectivity at the level of transcriptional activation, the same 2 kb promoter sequence was also cloned into a modified luciferase expression cassette. This luciferase system features the luciferase coding sequence in the place of the E1 region in an adenoviral left shuttle plasmid. The promoter sequence was then cloned into restriction sites upstream of the luciferase coding sequence to drive luciferase expression. This modified reporter system more closely approximates the sequence context of the adenoviral ITR and packaging signal/enhancer that may influence the heterologous promoter activity. In addition, this system allows screening candidate promoters in a higher throughput scale, which accelerates the process of identifying selective motifs for further improvement of oncolytic vector selectivity and potency.

### Example 5

### Tumor Killing Selectivity And Potency By MTS Assay With The FEN1 Adenoviral Vector

The FEN1 oncolytic adenoviral vector was evaluated by an MTS assay according to manufacturer's instructions (CellTiter 96® AQᵤₑₒᵤₛ Assay by Promega, Madison, Wis.) for its selectivity on target vs. non-target and normal cell lines. Wildtype Ad5 was included in the experiment as a normalization factor. Cells were seeded in 96-well dishes in 90 μl volume one day prior to adenoviral infection. The next day, adenoviruses were diluted serially in the appropriate growth media and 10 μl of each dilution is added to the wells. Cells were exposed to virus for seven to ten days, after which an MTS cytotoxicity assay (CellTiter 96® AQᵤₑₒᵤₛ Non-Radioactive Cell Proliferation Assay (Promega, Madison, Wis.)) was performed according to the manufacturer's instructions. Absorbance values are expressed as a percent of uninfected control and plotted versus vector dose. A sigmoidal dose-response curve is fit to the data and a lethal dose-50 percent (LD₅₀) value was calculated for each replicate using GraphPad Prism software, version 3.0.

In evaluating the selectivity of an oncolytic vector in vitro, comparison with a wildtype control such as Ad5 helps control for potential differences between cell lines such as transduction efficiency. Selectivity for tumor cell lines can be represented mathematically by a “selectivity index” value. In the current example, a selectivity index value for a vector is the cytotoxicity of an oncolytic vector relative to Ad5 on tumor target versus non-target or normal cells. A selectivity index value above “1” is defined as having tumor cell selectivity. The higher the value, the better the selectivity. Tumor-killing selectivity is calculated based on the following equation:

$$Selectivity Index = \frac{{LD}_{50} Ad 5 target . tumor \div {LD}_{50} OV target . tumor}{{LD}_{50} Ad 5 nontarget . norma 1 \div {LD}_{50} OV nontarget . normal}$$

FEN1 was originally thought to be a colon cancer-selective gene by expression profiling. However, it was found later to be selectively expressed in various cancer tissue types including lung, breast, gastric and ovarian tumors in the database. A rt-PCR experiment carried out as described above also characterized the gene expression in multiple tumor cell lines (Table 3). By MTS assay, the selectivity indices for the FEN1 adenoviral vector were calculated for colon tumor cell lines SW620 and HT29; small cell lung carcinoma (SCLC) tumor cell lines H69 and H446; and prostate tumor cell lines C4-2 and PC3M2AC6 vs. the normal cell lines HRE, HAEC and Wi38. The selectivity index values indicated that the FEN1 vector was selectively killing SCLC, colon and, with less selectivity, prostate tumor cell lines (Table 4). The relative LD₅₀ values for FEN1 adenoviral vector were in the range of 0.2 to 0.5 for both small cell lung carcinoma and colon tumor cell lines (Table 4), indicating a killing potency within the same order of magnitude as that of wildtype Ad5 on target tumor cells. Therefore, the FEN1 adenoviral vector has both high cancer-killing selectivity and potency.

TABLE 4
Relative LD₅₀ for FEN1 Vector in Various Cell Lines in an MTS
Assay and FEN1 Vector Selectivity Index in an MTS Assay.

| | Relative LD₅₀ | Selectivity Index (vs normal cell) | | |
|---|---|---|---|---|
| Cell culture | (FEN1 Vector) | (WI-38) | (HRE) | (HAEC) |

Tumor cell lines

| | | | | |
|---|---|---|---|---|
| SW620 | 0.268 | 2.6 | 14.0 | 8.8 |
| HT29 | 0.163 | 1.6 | 8.5 | 5.4 |
| H69 | 0.301 | 2.9 | 15.6 | 9.9 |
| H446 | 0.493 | 4.7 | 25.6 | 16.2 |
| PC3M2AC6 | 0.089 | 0.8 | 4.6 | 2.9 |
| C4-2 | 0.003 | 0.03 | 0.2 | 0.1 |

Normal cells

| | | | | |
|---|---|---|---|---|
| WI-38 | 0.105 | na | na | na |
| HAEC | 0.030 | na | na | na |
| HRE | 0.019 | na | na | na |

### Example 7

### Virus Production Assay

Virus production assays are known to one skilled in the art. One example is as follows. Cells are seeded at 10,000 cells per well in 96-well plates in 190 ul of the appropriate media one day prior to infection. Each adenovirus vector is diluted in 10 ul appropriate media to achieve 10 particles per cell (ppc) final concentration. Cells are infected with each of the vector by transferring the 10 ul vector solution into the 190 ul media on the plate. For primary infection, the infected cells are incubated at 37° C. in a humidified 5% C02 incubator for three days. Crude viral lysates (CVL) at day three are generated by freezing-thawing the 96-well plates for five cycles. The plates are then centrifuged at 2000 rpm for 10 min and the supernatant is used as the CVL for the secondary infection. Secondary infection is performed using A549 based S8 cells (Gorziglia et al. J Virol. 1996 June;70(6):4173-8) as the indicator cell line for adenovirus infection. The original CVL is serially diluted 1:10 in Richter's media supplemented with 5% FBS and transferred into each well using a robot, model Biomek 2000 (Beckman, Fullerton, Calif.). The infected cells are incubated at 37° C. and 5% CO2 for 10 to 14 days. The plates are scored by absorbance values derived from cytotoxicity (MTS) assay and TCID50 is calculated.

To determine selective viral production for tumor cells, each viral titer is normalized to a relative titer to Ad5 to allow the comparison among cell lines. The selectivity index is then calculated from the relative titer in tumor cells versus the relative titer in normal cells.

In an exemplary study carried out according to the above, vector production was determined on SW620, H69 and H446 tumor cell lines versus HRE and MRC5 normal cell lines. The initial infection, using Ad5, Ar13pAFenF and AddI312 vector at an MOI of 10 ppc was harvested at three days post-infection. The titer was then determined. Each initial infection was performed in triplicate and each CVL was analyzed in replicates of 12. The viral titers are shown in FIG. 1. In general, all of the cell lines were capable of supporting viral infection and production of progeny virus for the replication competent viral vectors. Among the vectors tested, wildtype Ad5 vector produced the highest viral titers. Replication-defective vector Ad dI312 produced low biological titers (165 to 210 pfu/ml in tumor cell lines and 100 to 830 pfu/ml in the normal cell lines), which was at the limit of detection for this assay.

In summary, a cancer specific FEN1 promoter sequence has been identified and the tumor-specific oncolytic effect on target tumor cell lines has been verified.

The selectivity index was determined as described above. Table 5 shows that the Ar13pAFenF selectivity index for H69 and H446 was >100 over normal HRE cells. Therefore, this vector was selectively produced in tumor cell lines.

TABLE 5
Vector production: Selectivity of Ar13pAFenF vector

| | SW620 | H69 | H446 |
|---|---|---|---|
| MRC-5 | 2.5 | 4.1 | 3.5 |
| HRE | 79.3 | 130.8 | 111.9 |

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

Description Of The Sequences In The Sequence Listing

The Sequence Listing associated with the instant disclosure is hereby incorporated by reference into the instant disclosure. The following is a description of the sequences contained in the Sequence Listing:

| SEQ ID | DESCRIPTION |
|---|---|
| SEQ ID | 1992 bp fragment of the 2259 bp fragment |
| NO: 1 | of the human FEN1 TRE. |
| SEQ ID | 239 bp fragment of the human telomerase |
| NO: 2 | reverse transcriptase (hTERT) TRE. |
| SEQ ID | 245 bp fragment of the human TERT TRE. |
| NO: 3 | |
| SEQ ID | PCR primer 5′-GCAAGAAGGCCACAGAGGTACT-3′ |
| NO: 4 | |
| SEQ ID | PCR primer 5′-GATTGCCAGGTGAACATCACCATC-3′ |
| NO: 5 | |
| SEQ ID | PCR primer 5′-CATGCTGCTAGCCATGCGGTTATCAAGGA |
| NO: 6 | GCC-3′ |
| SEQ ID | PCR primer 5′-TTGGATATCGACGTTCAGCCGCCTTC |
| NO: 7 | CAA-3′ |
| SEQ ID | 270 bp fragment of the E2F promoter |
| NO: 8 | |

2259 bp fragment of the human FEN1 TRE

CATGCGGTTATCAAGGAGCCTGGTGCTGCCGTGAAACAGAGGCTGATTTT
AGCCCGGAAATGTAGCTGCAGATCAATGGCCCTTATTAGCATTTTCTGAG
GCCAATAATCTGACCACTATGAAAACGTGACTAAAGGTACGAACTCTCTG
CCTGAGAAAAACCACATACAAGAAAAAGTTTGCCTACAATTTCCGGAGCT
TTGTGGACCAGTGTCTATAGACACCAAGCTGAGAACCCCCGCTATAAGTC
ACTGACTGGTGGTACCCAGATCTCAATATCTTTTTTTTTTGACGGAGTCT
CATTTTTTGGACGGCGTCTCACTCTGTCGCCCGGGCTGGAGGGCAGTGGC
ACGATCTCGGCTCACTGCAACCTCTGCCTCCCGGGTTCTAGAGATTCTCA
TACCTCAGCCTCTCGAGTAGCTGGGACTATAGGATTACAGGTGCGCACCA
CCACATCTAATTTTTGTATTTTTAGTAGAGATGGGGTTTTGCCATGCTGG
CCAGGATGGTCTTGAATTCCTGACCTCAGGTGATCTGCCTGCCTCGGCCT
CCCAAAGTACTGAGATTACAGGTGTGAGTTGCCGCGCCCAGGCTCAATTT
TTTTTTTTTTCCAGACAGTCTTGCTCTATCGCCCAGGCTGGAGTGCCTGG
AGTGCAGTGGTGCCAACTCGGCTCACTGCAAGCTCCGCCTTCTGGGTTCA
AGTGATTATCCTGCCTCAGCCTCCCGAGCAGCTGGGATTACAGGTGTGAA
CCACCATGCCCGGCTAATTTTTTGTATTTTTAGGAGAGACAGGGTTTCAC
CTTGCTGGCCAGGCTGGTCTTGAACTTCTGACCTCCTGATCCGCTCGCCT
CAGCCTCCCAAAGTGCTGGGATTACAGGAGTGAACCACCGCGCCTGGCCC
TCAATTTCTAATTCAGTATTTTCCTCACTACCTATGCTATTATGGAATCT
TGTGAGCTATGGTCAAGACATTCAAGTTCTGGTTCTGAGTAATCTGAGTC
TGAGTAAAGCGACTGTAATATCTATTTCACAGAACTGAAAAATAAGAAAG
ATGATGAATCAAAGCATCTAGTGCCTAGCAGGGAGTATTTTGCTCAACAG
GTATTTGCTTCCTTCCTAAGGCTGTAGGGAAGATGATGAGATAATGTCTT
TTATGAAAGAGGGCTGTAAACGTAAAGATCTGTACAAATGTTAACTTCAT
TGTCACCGGTCAGCCAATGCTTCTAAAATCCAGAACATAACAACTCTAGA
GAAGTAAACTGCCCCCATTGTTCTGAGACACTGGAATTCAATTCAGTAAA
CAATCACGGCCCCCTTCCCCCAAAATGATAAAGACAATCACTGCCATTTA
TTGAGCTTCCAATTACGGGCCCTCTGTTTGGCACTGAGAATACAAAGATG
AATAGACATCATCCCAGAGCTAGATGCGCGTCAGACGGTGGTCACTAGGA
GGCGTGGCCGAAAACAAAGAAGTCCATGGAACGTGGCCAGAGATCTGTAC
AGAGGCTGTGGGCGCTCCTAGGAAAGTCTGGCCAAGTGCCTGAGAGTTGG
AAGTGCTTCACCAATAAACATTTGCCCAGGGCATTGTAGGATGGGCACGG
GTTCGGCAGAAGAACTTTCCAAATAAAGATAACACACCACCGATAACAGA
GATATACAAACTGGAAGGTATTCAAAATTCGCCCCACGCCTCTCGCCCTT
AGAAATCGCGAGCTGAGAAACCTAAGGAGTTCATGGCAAGGGGCTTCCCC
CTTCCCCACCCTTCAGCCCAAGCCGGAGGTTCCAGGAGCGTCTAGCCCTC
TGGATCTCCGGCGTCTGAGGAGATAAGCGCGGTGTGGGTCAGACCCCGAG
GGGTCCTCGCATCTCCGTCTGGAACTCCCCTCAACGCTCTCACCATTTTG
CCCCGCGAAGGCTAATCCGCCGCTCCGCCACCGGAAGAACACGTCGACAG
GAGCAGGCGCCTAGCACAACCGGAAAAGGAAGTGCCTCCGGCGCAAGTGG
CATTGAGGGACTTGTAGTCCTGCGATTTCGGGTGTAGAGGGAGCAGGGGC
CTGCGGGGACCTGGTGTGGGTGGAGTGGGGACAAGCGGTGGAGAAGGGTA
CGCCAGGGTCGCTGAGAGACTCTGTTCTCCCTGGAGGGACTGGTTGCCAT
GAGAGCAGCCGTCTGAGGGGACGCAGCCTGCACTACGCGCCCCAAGAGGC
TGTGCGTGGCGAGCAGGTCACGTGACGGGAGCGCGGGCTTTGGAAGGCGG
CTGAACGTC

What is claimed is:

1. An isolated nucleic acid sequence comprising a cancer specific transcriptional regulatory element (TRE) derived from the sequence upstream of the translational start codon for a FEN1 gene, wherein said TRE is specific for cancer cells.

2. The isolated nucleic acid sequence according to claim 1, wherein said cancer cells are colon cancer cells.

3. The isolated nucleic acid sequence according to claim 1, wherein said TRE is a human TRE.

4. The isolated nucleic acid sequence according to claim 2, wherein said TRE is the FEN1 TRE presented as SEQ ID NO:1.

5. The isolated nucleic acid sequence according to claim 4, wherein said TRE is a functional fragment of the FEN1 TRE presented as SEQ ID NO:1.

6. A replication competent adenovirus vector comprising a cancer specific transcriptional regulatory element (TRE) derived from the sequence upstream of the translational start codon for a FEN1 gene, wherein said adenovirus vector selectively replicates in cancer cells.

7. A replication competent adenovirus vector according to claim 6, wherein said TRE is the FEN1 TRE presented as SEQ ID NO:1.

8. The adenovirus vector according to claim 7, wherein said adenovirus vector has a first adenovirus gene essential for replication under transcriptional control of said FEN1 TRE.

9. The adenovirus vector according to claim 8, wherein said first adenovirus gene essential for replication is an early gene selected from the group consisting of E1a, E1b and E4.

10. The adenovirus vector according to claim 9, wherein the adenoviral vector comprises first and second adenoviral genes co-transcribed under transcriptional control of said FEN1 TRE.

11. The adenovirus vector according to claim 10, further comprising an IRES.

12. The adenovirus vector according to claim 10, further comprising a self-processing cleavage sequence.

13. The adenovirus vector according to claim 9, further comprising a transgene.

14. The adenovirus vector according to claim 9, further comprising a second adenovirus gene essential for replication under transcriptional control of a colon cancer specific PRL-3 TRE.

15. The adenovirus vector according to claim 14, wherein said second adenovirus gene essential for replication is an early gene selected from the group consisting of E1a, E1b and E4.

16. The adenovirus vector according to claim 9, further comprising a second adenovirus gene essential for replication under transcriptional control of a TERT-TRE or an E2F-TRE.

17. The adenovirus vector according to claim 16, wherein said second adenovirus gene essential for replication is an early gene selected from the group consisting of E1a, E1b and E4

18. An isolated host cell comprising the adenovirus vector of claim 4.

19. An isolated host cell comprising the adenovirus vector of claim 9.

20. An isolated host cell comprising the adenovirus vector of claim 15.

21. An isolated host cell comprising the adenovirus vector of claim 17.

22. A composition comprising the adenovirus vector of claim 4 and a pharmaceutically acceptable excipient.

23. A composition comprising the adenovirus vector of claim 9 and a pharmaceutically acceptable excipient.

24. A composition comprising the adenovirus vector of claim 15 and a pharmaceutically acceptable excipient.

25. A composition comprising the adenovirus vector of claim 17 and a pharmaceutically acceptable excipient.

26. The adenovirus vector according to claim 13, wherein the transgene is cytotoxic.

27. The adenovirus vector according to claim 13, wherein the transgene is a cytokine.

28. The adenovirus vector according to claim 9, further comprising a polynucleotide encoding adenoviral death protein (ADP).

29. An adenovirus vector according to claim 13, further comprising a polynucleotide encoding adenoviral death protein (ADP).

30. The adenovirus vector of claim 27, wherein said cytokine is GM-CSF gene.

31. The adenovirus vector according to claim 13, wherein said transgene is under transcriptional control of a colon cancer specific PRL-3 TRE.

32. The adenovirus vector according to claim 13, wherein said transgene is under transcriptional control of a TERT-TRE or an E2F-TRE.


SEQUENCE LISTING (8 sequences)

SEQ ID NO: 1 - DNA, 2259 bp, Homo sapiens
catgcggttatcaaggagcctggtgctgccgtgaaacagaggctgattttagcccggaaatgtagctgcagatcaatggcccttattagcattttctgaggccaataatctgaccactatgaaaacgtgactaaaggtacgaactctctgcctgagaaaaaccacatacaagaaaaagtttgcctacaatttccggagctttgtggaccagtgtctatagacaccaagctgagaacccccgctataagtcactgactggtggtacccagatctcaatatcttttttttttgacggagtctcattttttggacggcgtctcactctgtcgcccgggctggagggcagtggcacgatctcggctcactgcaacctctgcctcccgggttctagagattctcatacctcagcctctcgagtagctgggactataggattacaggtgcgcaccaccacatctaatttttgtatttttagtagagatggggttttgccatgctggccaggatggtcttgaattcctgacctcaggtgatctgcctgcctcggcctcccaaagtactgagattacaggtgtgagttgccgcgcccaggctcaatttttttttttttccagacagtcttgctctatcgcccaggctggagtgcctggagtgcagtggtgccaactcggctcactgcaagctccgccttctgggttcaagtgattatcctgcctcagcctcccgagcagctgggattacaggtgtgaaccaccatgcccggctaattttttgtatttttaggagagacagggtttcaccttgctggccaggctggtcttgaacttctgacctcctgatccgctcgcctcagcctcccaaagtgctgggattacaggagtgaaccaccgcgcctggccctcaatttctaattcagtattttcctcactacctatgctattatggaatcttgtgagctatggtcaagacattcaagttctggttctgagtaatctgagtctgagtaaagcgactgtaatatctatttcacagaactgaaaaataagaaagatgatgaatcaaagcatctagtgcctagcagggagtattttgctcaacaggtatttgcttccttcctaaggctgtagggaagatgatgagataatgtcttttatgaaagagggctgtaaacgtaaagatctgtacaaatgttaacttcattgtcaccggtcagccaatgcttctaaaatccagaacataacaactctagagaagtaaactgcccccattgttctgagacactggaattcaattcagtaaacaatcacggcccccttcccccaaaatgataaagacaatcactgccatttattgagcttccaattacgggccctctgtttggcactgagaatacaaagatgaatagacatcatcccagagctagatgcgcgtcagacggtggtcactaggaggcgtggccgaaaacaaagaagtccatggaacgtggccagagatctgtacagaggctgtgggcgctcctaggaaagtctggccaagtgcctgagagttggaagtgcttcaccaataaacatttgcccagggcattgtaggatgggcacgggttcggcagaagaactttccaaataaagataacacaccaccgataacagagatatacaaactggaaggtattcaaaattcgccccacgcctctcgcccttagaaatcgcgagctgagaaacctaaggagttcatggcaaggggcttcccccttccccacccttcagcccaagccggaggttccaggagcgtctagccctctggatctccggcgtctgaggagataagcgcggtgtgggtcagaccccgaggggtcctcgcatctccgtctggaactcccctcaacgctctcaccattttgccccgcgaaggctaatccgccgctccgccaccggaagaacacgtcgacaggagcaggcgcctagcacaaccggaaaaggaagtgcctccggcgcaagtggcattgagggacttgtagtcctgcgatttcgggtgtagagggagcaggggcctgcggggacctggtgtgggtggagtggggacaagcggtggagaagggtacgccagggtcgctgagagactctgttctccctggagggactggttgccatgagagcagccgtctgaggggacgcagcctgcactacgcgccccaagaggctgtgcgtggcgagcaggtcacgtgacgggagcgcgggctttggaaggcggctgaacgtc

SEQ ID NO: 2 - DNA, 239 bp, Homo sapiens
cgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgc

SEQ ID NO: 3 - DNA, 245 bp, Homo sapiens
ccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 4 - DNA, 22 bp, Artificial Sequence
  Description of Artificial Sequence Synthetic primer
gcaagaaggccacagaggtact

SEQ ID NO: 5 - DNA, 24 bp, Artificial Sequence
  Description of Artificial Sequence Synthetic primer
gattgccaggtgaacatcaccatc

SEQ ID NO: 6 - DNA, 32 bp, Artificial Sequence
  Description of Artificial Sequence Synthetic primer
catgctgctagccatgcggttatcaaggagcc

SEQ ID NO: 7 - DNA, 29 bp, Artificial Sequence
  Description of Artificial Sequence Synthetic primer
ttggatatcgacgttcagccgccttccaa

SEQ ID NO: 8 - DNA, 270 bp, Homo sapiens
tggtaccatccggacaaagcctgcgcgcgccccgccccgccattggccgtaccgccccgcgccgccgccccatcccgcccctcgccgccgggtccggcgcgttaaagccaataggaaccgccgccgttgttcccgtcacggccggggcagccaattgtggcggcgctcggcggctcgtggctctttcgcggcaaaaaggatttggcgcgtaaaagtggccgggactttgcaggcagcggcggccgggggcggagcgggatcgagccctcg